(19) United States
(12) Patent Application Publication
(10) Pub. No.: US 2022/0290244 A1
(43) Pub. Date: Sep. 15, 2022

(54) METHOD FOR SCREENING A SUBJECT FOR A CANCER

(71) Applicants: INSERM (Institut National de la Santé et de la Recherche Médicale), PARIS (FR); Institut Régional du Cancer de Montpellier, MONTPELLIER (FR); Université de Montpellier, MONTPELLIER (FR)

(72) Inventor: Alain THIERRY, MONTPELLIER (FR)

(21) Appl. No.: 17/631,908

(22) PCT Filed: Jul. 31, 2020
(86) PCT No.: PCT/EP2020/071637

§ 371 Date: Feb. 1, 2022

(30) Foreign Application Priority Data
Aug. 2, 2019 (EP) 19306003.5

(51) Int. Cl. C12Q 1/6886

(52) U.S. Cl. CPC C12Q 1/6886

(57) ABSTRACT

The present invention relates to the field of cancer diagnostic. Here, the inventors observed by using conventional DSP sequencing method that size profile of double stranded DNA fragments obtained from cell free nucleic acids (cfDNA) may discriminate cfDNA from healthy and cancer derived subjects as previously observed (Jiang et al). Contrary to the prior art, the inventors determined specific double stranded DNA fragments or range of fragments and showed that these the number of these specific fragments are different between healthy and cancerous subjects. The number of double stranded DNA fragments as quantified from CfDNA are rather lower or higher when derived from healthy subject than from cancer subject. The invention provides description of calculation or biomarker from the identification of specific DNA fragments, specific ratios for different size or range of double stranded DNA fragment towards discriminating cancer to healthy plasma. The present invention relates to a method for screening a subject for a cancer by determining the level of double stranded DNA fragments in a sample.

### FIELD OF THE INVENTION

The present invention relates to a method for screening a subject for a cancer by determining the level of double stranded DNA fragments in a sample.

### BACKGROUND OF THE INVENTION

Analysis of circulating cell-free DNA (cfDNA) undoubtedly represents a breakthrough in the diagnostic field, and in predictive, preventive and personalized medicine. The potential of this newly-identified source of biological information has attracted the attention of researchers and clinicians in numerous fields. One of the emerging cfDNA feature when optimizing cfDNA analysis is the fragmentation. Fragment size of ˜180 bp corresponding to the length of the DNA molecule wrapped around a mononucleosome was initially thought to be the lowest size to be found in the blood stream. The inventors first showed that cfDNA is highly fragmented down to size below 100 bp (1). They also demonstrated that cancer patient derived cfDNA are shorter than that of healthy individuals fueling research on fragmentation to better discriminate cfDNA origins (i.e., (2)). The cfDNA structure and size are intimately associated because of the high nuclease sensitivity of the naked DNA molecule. Consequently, these two features are highly scrutinized in the recent years to improve knowledge on cfDNA release, to improve cfDNA detection and to evaluate cfDNA potential in discriminating cfDNA tissue/cells of origin for enlarging cfDNA diagnostic power.

Circulating cell-free DNA (cfDNA) can be present in the form of protein-associated DNA fragments or extracellular vesicles, in the physiological circulating fluids of healthy and diseased individuals as recently reviewed(3, 4). cfDNA is derived not only from genomic DNA but also from extrachromosomal mitochondrial DNA. While various clinical applications of cfDNA are currently progressing(5, 6), the identification of structural characteristics remains under investigation.

Higher fragmentation has been found in cfDNA from cancer patients (1), from tumor cells (7), and in the fetal fraction(8, 9). Efforts are ongoing either to increase analytical sensitivity by specifically targeting a fragment size population, or to stratify individuals, for example, in cancer screening. The examination of fragmentation level is also a good quality control parameter for estimating the stability and integrity of cfDNA extracts.

The inventors previously characterized cfDNA fragment length distribution from plasma of cancer patients (10, 35, 36 and patent applications WO2012028746 and WO/2019/110750). In a blinded study, they demonstrated that circulating tumor DNA from cancer patients showed a similar size profile using either whole genome deep sequencing from single strand library preparation (SSP) or using a Q-PCR based method, revealing a higher proportion of shorter cfDNA fragments (below 80 bp) not readily detectable by standard double-stranded DNA library preparation (DSP) protocols (as previously shown by Underhill et al (11)). This showed the higher capacity of single strand DNA template analysis to detect cfDNA fragments, and revealed that cfDNA molecules, which are initially packed in chromatin, are released from cells and then dynamically degraded in blood, both within and between nucleosomes or transcription factor-associated subcomplexes. In addition, this study revealed that cfDNA size distribution obtained from the conventional WGS from a double-stranded DNA library should be distinguished from that obtained with Q-PCR or WGS from a single-stranded DNA library which use single-strand DNA as a first template. In addition to explaining the ctDNA size profile controversy, this study buttress the notion that ctDNA pattern is predominantly guided by mononucleosome pattern and that nicks occur within the cfDNA sequence packed in the mononucleosome patients (WO/2019/110750, Sanchez, npj genomic medicine, 2018). They showed by using sequencing that a subtle variation in cfDNA size profile exists in case of cancer patient plasma which is proportional to the concentration of mutant (malignant or tumor-derived) cfDNA fragments. They consequently highlighted some calculation made from specific sizes, size ranges or ratio of provided strong discrimination between cancer and healthy individuals. They also described that fragmentation varied upon the position in the cfDNA genome background from cancer patient plasma, in contrast to healthy individuals, and higher fragmentation was specifically observed in several chromosomes, providing possibility to discriminate cancer plasma with healthy plasma by examining fragmentation level per chromosome.

### SUMMARY OF THE INVENTION

Here, the inventors observed by using conventional DSP sequencing method that size profile of double stranded DNA fragments obtained from cell free nucleic acids (cfDNA) may discriminate cfDNA from healthy and cancer derived subjects as previously observed (Jiang et al). Contrary to the prior art and notably Cristiano S et al, here the inventors determined specific double stranded DNA fragments or range of fragments and showed that these the number of these specific fragments are different between healthy and cancerous subjects. The number of double stranded DNA fragments as quantified from CfDNA are rather lower or higher when derived from healthy subject than from cancer subject. The invention provides description of calculation or biomarker from the identification of specific DNA fragments, specific ratios for different size or range of double stranded DNA fragment towards discriminating cancer to healthy plasma. First, the invention resides from determining those calculation/biomarkers from comparing cancer to healthy cfDNA lengths profiles. Second, the invention relates calculation/biomarkers from comparing the cumulative size frequency of the difference of healthy and cancer cfDNA size profile by sequencing. Third, discrimination between healthy and cancer plasma can be achieved by directly comparing in the same plasma sample the former cumulative size frequency of the difference between healthy and cancer cfDNA size profile by double-stranded DNA sequencing to the cumulative size frequency of the difference of healthy and cancer cfDNA size profile by single-stranded DNA sequencing (which artificially detected non-natural single strand DNA fragments through an initial denaturation step of the cfDNA extract of the biological fluids). Fourth, discrimination between healthy and cancer plasma can be achieved by comparing the number of fragments per chromosome (or fragmentation level) showing high discrepancy in identified chromosome.

These values are sufficiently and significantly different to be used as values to determine whether a human subject may have cancer or not as a screening test.

These values are sufficiently and significantly different to be used as values to determine whether a human subject may have cancer or not as a screening test.

Thus, the present invention relates to a method for screening a subject for a cancer by determining the level of double stranded DNA fragments in a sample. Particularly, the invention is described by its claims.

### DETAILED DESCRIPTION OF THE INVENTION

### Analyse of Double Stranded DNA Fragments

A first aspect of the invention relates to a method for screening a subject for a cancer comprising the steps of:
i. extracting the cell free nucleic acids (cfDNA) from a sample obtained from the subject;
ii. determining the level of at least one double stranded DNA fragment having a length between 20 to 440 base pairs (bp);
iii. comparing the level determined at step ii) with a predetermined reference value and;
iv. concluding that the subject suffers from a cancer when the level determined at step iii) differ from the predetermined reference value.

Particularly the level of at least one double stranded DNA fragment determined at step ii) have a length between 20 to 400 base pairs.

Thus, the invention relates to a method for screening a subject for a cancer comprising the steps of:
i. extracting the cell free nucleic acids (cfDNA) from a sample obtained from the subject;
ii. determining the level of at least one double stranded DNA fragment having a length between 20 to 400 base pairs (bp);
iii. comparing the level determined at step ii) with a predetermined reference value and;
iv. concluding that the subject suffers from a cancer when the level determined at step iii) differ from the predetermined reference value.

Particularly the level can be calculated with double stranded DNA fragment having a length of 120+/−5, 130+/−5 or 134+/−5 bp.

More particularly, the level can be calculated with double stranded DNA fragment having a length of 115, 116, 117, 118, 119, 120, 121, 122, 123, 124, 125, 126, 127, 128, 129, 130, 131, 132, 133, 134, 135, 136, 137, 138 or 139.

More particularly, the level can be calculated with double stranded DNA fragment having a length of 120, 130 or 134 bp.

More particularly the level can be calculated with double stranded DNA fragment having a length of 145 or 166 bp.

In another particular embodiment, the level can be calculated with several (or a group of) double stranded DNA fragments. Particularly the group of fragments can have a length range of 119 to 120 or 40 to 250 bp.

Particularly the group of fragments can have a length range of 40 to 160 bp, 40 to 120 bp or 40 to 80 bp.

Particularly the level can be calculated with double stranded DNA fragment having a length of 80 to 120 or 130 to 160 bp.

Particularly the level can be calculated with double stranded DNA fragment having a length of 30 to 220 or 30 to 145 bp.

Particularly the level can be calculated with double stranded DNA fragment having a length of 30 to 145 bp.

As used herein the term “sample” refers to any biological sample obtained from the subject that is liable to contain cell free nucleic acids. Typically, samples include but are not limited to body fluid samples, such as blood, ascite, urine, amniotic fluid, feces, saliva or cerebrospinal fluids. In some embodiments, the sample is a blood sample. By “blood sample” it is meant a volume of whole blood or fraction thereof, e.g., serum, plasma, etc. In one embodiment, the sample consists in culture supernatant of cells, embryo or organoid. Any methods well known in the art may be used by the skilled artisan in the art for extracting the free cell nucleic acid from the prepared sample. For example, the method described in the EXAMPLE may be used.

As used herein, the term “subject” denotes a mammal. Typically, a patient according to the invention refers to any subject (preferably human) afflicted with a cancer. The term “subject” also refers to a subject with no cancer.

As used herein, the term “double stranded DNA fragment” denote double stranded (compared to single stranded) fragment of DNA which can have different size of nucleic acids.

According to the invention, the term “length” or “size” are used equivalently and have the same meaning.

As used herein the terms the “level of at least one double stranded DNA fragment having a length between 20 to 400 bp” denote the quantity (or concentration) of at least one double stranded DNA fragment of a specific size (e.g. of a specific length of nucleic) whatever the sequence of the fragments. For example the terms the “level of a double stranded DNA fragment having a length of 50 bp” denotes the level (quantity or concentration) of all the double stranded DNA fragments having a length of 50 bp whatever the sequence of the fragments of 50 bp.

According to the invention, as used herein, the “level” means quantity, number or concentration of a fragment. Particularly, the term “level” also denotes the number (quantity) of sequencing reads, the fragment number (or quantity) or a percentage of the total of fragment number.

As used herein the term “nucleic acid” has its general meaning in the art and refers to refers to a coding or non-coding nucleic sequence. Nucleic acids include DNA (deoxyribonucleic acid) and RNA (ribonucleic acid) nucleic acids. Example of nucleic acid thus include but are not limited to DNA, mRNA, tRNA, rRNA, tmRNA, miRNA, piRNA, snoRNA, and snRNA. Nucleic acids thus encompass coding and non-coding region of a genome (i.e. nuclear or mitochondrial).

As used herein, the term “nuclear nucleic acid” has its general meaning in the art and refers to a nucleic acid originating from the nucleus of cell. The term nuclear nucleic acid encompasses all forms of the nucleic acids excepting those originating from the mitochondria. The term nuclear nucleic acid is thus defined in opposition to the term “mitochondrial nucleic acid”. Mitochondria are indeed structures within cells that convert the energy from food into a form that cells can use. Although most DNA is packaged in chromosomes within the nucleus, mitochondria also have a small amount of their own DNA. This genetic material is known as “mitochondrial DNA” or “mtDNA”. In humans, mitochondrial DNA spans about 16,500 DNA building blocks (base pairs), representing a small fraction of the total DNA in cells. Mitochondrial DNA contains 37 genes, all of which are essential for normal mitochondrial function: ATP6; ATP8; COX1; COX2; COX3; CYTB; ND1; ND2; ND3; ND4; ND4L; ND5; ND6; RNR1, RNR2 TRNA; TRNA; TRNC; TRND; TRNE; TRNF; TRNG; TRNI; TRNK; TRNL1; TRNL2; TRNM; TRNN; TRNN; TRNP; TRNQ; TRNR; TRNS1; TRNS2; TRNT; TRNV; TRNW; and TRNY. Genes encoding for NADH dehydrogenase (complex I) include MT-ND1, MT-ND2, MT-ND3, MT-ND4, MT-ND4L, MT-ND5, and MT-ND6. Genes encoding for Coenzyme Q-cytochrome c reductase/Cytochrome b (complex III) include MT-CYB. Gene encoding for cytochrome c oxidase (complex IV) include MT-CO1, MT-CO2, MT-CO3. Gene enconding for ATP synthase (complex V) include MT-ATP6, and MT-ATP8. Gene encoding for humanin include MT-RNR2 (encoding both ribosomal 16S and humanin). MT-RNR1 and MT-RNR2 genes providing instruction to produce ribosomal 12S and 16S respectively. The 22 species of mitochondrial tRNAs (mt tRNAs) encoded by mtDNA involved in mitochondrial protein synthesis machinery. Human mitochondrial DNA (mtDNA) has three promoters, H1, H2, and L (heavy strand 1, heavy strand 2, and light strand promoters). Mitochondrial genome also comprises control regions or d-loop sequences. Mitochondrial nuclear acids are known per se by the skilled person (e.g. NCBI Reference Sequence: NC_012920.1, SEQ ID NO:1). Thirteen of these genes provide instructions for making enzymes involved in oxidative phosphorylation. Oxidative phosphorylation is a process that uses oxygen and simple sugars to create adenosine triphosphate (ATP), the cell's main energy source. The remaining genes provide instructions for making molecules called transfer RNA (tRNA) and ribosomal RNA (rRNA), which are chemical cousins of DNA. These types of RNA help assemble protein building blocks (amino acids) into functioning proteins.

By “cell free nucleic acid” or “cfDNA” it is meant that the nucleic acid is released by the cell and present in the sample. In some embodiments, the cell free nucleic acid is circulating cell-free DNA (ccfDNA) and it is easy and routine for one of ordinary skill in the art to distinguish mitochondrial ccf nucleic acids” or “mitochondrial ccfDNA” from “nuclear ccfDNA”. Actually, mitochondrial ccfDNA encompasses any DNA mitochondrial nucleic acid and in opposition nuclear ccfDNA encompasses any DNA nuclear nucleic acid.

As used herein, the term “cancer” has its general meaning in the art and includes, but is not limited to, solid tumors and blood borne tumors. The term cancer includes diseases of the skin, tissues, organs, bone, cartilage, blood and vessels. The term “cancer” further encompasses both primary and metastatic cancers. Examples of cancers include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyo sarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In some embodiments, the subject suffers from a colorectal cancer, more particularly a metastatic colorectal cancer.

According to the methods of the invention, the cell free nucleic acids are cell free DNA (cfDNA) or circulating cell free DNA (ccfDNA).

According to the methods of the invention, the level of the double stranded DNA fragments of the level of group of double stranded DNA fragments can be made by Q-PCR based method or by sequencing methods. Particularly, the sequencing methods are based on a single stranded DNA library preparation.

According to the invention, the double stranded DNA fragments, can be from a group of genes, same gene or the same exon.

According to the invention, the double stranded DNA fragments can be a nuclear or a mitochondrial DNA.

According to the invention, to amplify the double stranded DNA fragments, 1 set of 3 primers can be used.

As used herein, the term “primer” refers to an oligobp, whether occurring naturally as in a purified restriction digest or produced synthetically, which is capable of acting as a point of initiation of nucleic acid sequence synthesis when placed under conditions in which synthesis of a primer extension product which is complementary to a nucleic acid strand is induced, i.e. in the presence of different bp triphosphates and a polymerase in an appropriate buffer (“buffer” includes pH, ionic strength, cofactors etc.) and at a suitable temperature. Typically, a primer has a length of 10; 11; 12; 13; 14; 15; 16; 17; 18; 19; 20; 21; 22; 23; 24; 25; 26; 27; 28; 29; or 30 bp. One or more of the bp of the primer can be modified for instance by addition of a methyl group, a biotin or digoxigenin moiety, a fluorescent tag or by using radioactive bp. A primer sequence need not reflect the exact sequence of the template. For example, a non-complementary bp fragment may be attached to the 5′ end of the primer, with the remainder of the primer sequence being substantially complementary to the strand. Primers are typically labelled with a detectable molecule or substance, such as a fluorescent molecule, a radioactive molecule or any others labels known in the art. Labels are known in the art that generally provide (either directly or indirectly) a signal. The term “labelled” is intended to encompass direct labelling of the probe and primers by coupling (i.e., physically linking) a detectable substance as well as indirect labeling by reactivity with another reagent that is directly labeled. Examples of detectable substances include but are not limited to radioactive agents or a fluorophore (e.g. fluorescein isothiocyanate (FITC) or phycoerythrin (PE) or Indocyanine (Cy5)).

According to the invention, a first double stranded DNA fragment having a length between 20 to 440 bp can be compared to another double stranded DNA fragment having a length between 20 to 440 bp. In particular, a first double stranded DNA fragment having a length between 20 to 400 bp can be compared to another double stranded DNA fragment having a length between 20 to 400 bp. More, the level of a group of double stranded DNA fragments can be determined and compare to another group of double stranded DNA fragments. Thus, according to the invention, when two levels or group of levels (as used in the invention) are compared between each other, simple comparison of the levels, difference between the levels or ratio of the levels can be done (see the examples).

Thus, in one embodiment, the method for screening a subject for a cancer comprises the steps of:
i. extracting the cell free nucleic acids from a sample obtained from the subject;
ii. determining the level of a first double stranded DNA fragment having a length between 20 to 440 bp;
iii. determining the level of a second double stranded DNA fragment having a length between 20 to 440 bp;
iv. calculating the ratio of the level determined at step ii) to the level determined at step iii) or alternatively the ratio of the level determined at step iii) to the level determined at step ii;
v. comparing the ratio determined at step iv) with a predetermined corresponding reference value and;
vi. concluding that the subject suffers from a cancer when the ratio determined at step iv) differ from the predetermined corresponding reference value.

In some embodiment, the level of the first double stranded DNA fragment determined at step ii) have a length between 20 to 400 base pairs, and the level of the second double stranded DNA fragment determined at step iii) have a length between 20 to 400 base pairs.

Thus, in one embodiment, the method for screening a subject for a cancer comprises the steps of:
i. extracting the cell free nucleic acids from a sample obtained from the subject;
ii. determining the level of a first double stranded DNA fragment having a length between 20 to 400 bp;
iii. determining the level of a second double stranded DNA fragment having a length between 20 to 400 bp;
iv. calculating the ratio of the level determined at step ii) to the level determined at step iii) or alternatively the ratio of the level determined at step iii) to the level determined at step ii;
v. comparing the ratio determined at step iv) with a predetermined corresponding reference value and;
vi. concluding that the subject suffers from a cancer when the ratio determined at step iv) differ from the predetermined corresponding reference value.

According to the invention, the ratio can be calculated by using specific length of bp like max peak (158 to 170)/130 bp, max peak (158 to 170)/137 bp or 307/322 bp.

According to the invention, the ratio can be calculated by using specific length of 166/145 bp.

In one embodiment, the method for screening a subject for a cancer comprises the steps of:
i. extracting the cell free nucleic acids from a sample obtained from the subject;
ii. determining the level of a first double stranded DNA fragment having a length between 20 to 440 bp;
iii. determining the level of a second double stranded DNA fragment having a length between 20 to 440 bp;
iv. calculating the difference with the level determined at step ii) to the level determined at step iii) or alternatively the difference of the level determined at step iii) to the level determined at step ii;
v. comparing the difference determined at step iv) with a predetermined corresponding reference value and;
vi. concluding that the subject suffers from a cancer when the difference determined at step iv) differ from the predetermined corresponding reference value.

In one embodiment, the first double stranded DNA fragment determined at step ii) have a length between 20 to 400 bp, and the level of a second double stranded DNA fragment determined at step iii) have a length between 20 to 400 bp.

Thus, in one embodiment, the method for screening a subject for a cancer comprises the steps of:
i. extracting the cell free nucleic acids from a sample obtained from the subject;
ii. determining the level of a first double stranded DNA fragment having a length between 20 to 400 bp;
iii. determining the level of a second double stranded DNA fragment having a length between 20 to 400 bp;
iv. calculating the difference with the level determined at step ii) to the level determined at step iii) or alternatively the difference of the level determined at step iii) to the level determined at step ii;
v. comparing the difference determined at step iv) with a predetermined corresponding reference value and;
vi. concluding that the subject suffers from a cancer when the difference determined at step iv) differ from the predetermined corresponding reference value.

According to the invention, the length of the double stranded DNA use to calculate the difference can be between 20 to 400 bp. Particularly, the difference can be calculate between a length of 134 and 124 by or a length of 155 and 145 bp.

In another embodiment, the method of the invention comprises the steps of:
i. extracting the cell free nucleic acids from a sample obtained from the subject;
ii. determining the level of a first double stranded DNA fragment having a length inferior to the peak (i.e. short size double stranded DNA fragment);
iii. determining the level of a second double stranded DNA fragment having a length superior or equal to the peak (i.e long size double stranded DNA fragment);
iv. calculating the ratio of the level determined at step ii) to the level determined at step iii) or alternatively the ratio of the level determined at step iii) to the level determined at step ii;
v. comparing the ratio determined at step iv) with a predetermined corresponding reference value and;
vi. concluding that the subject suffers from a cancer when the ratio determined at step iv) differ from the predetermined corresponding reference value.

As used herein, in this example the term “double stranded DNA fragment having a length inferior to the peak” denotes double stranded DNA fragment having a length inferior to the peak and superior or equal to 20 bp.

As used herein, in this example, the term “double stranded DNA fragment having a length superior to the peak” denotes double stranded DNA fragment having a length superior or equal to the peak and inferior or equal to 440 bp, and more particularly to 400 bp.

As shown in the examples, the inventors demonstrated that size profile of cfDNA fragments from a subject suffering from a cancer and from a healthy subject peaked at 166 or 167 bp. Theoretically, the peak corresponds to the size of cfDNA fragment associated with a chromatasome constituted of a nucleosome with Histone H1 which is 167 bp. Thus, our size profile measurement by both DSP-S or SSP-S strictly observed the theory. The peak (as used in the examples) denotes the maximal value of the size profile. Since, the peak can variate upon the method or device used to measure the length of the fragment, it can vary between a length of 158 to 170 bp or bp. Thus, the peak can correspond to fragments having a length of 158, 159, 160, 161, 162, 163, 164, 165, 166, 167, 168, 169 or 170 bp. These fragments (fragments having a length of 160, 161, 162, 163, 164, 165, 166, 167 or 168 bp) are also the most present (with the maximum level).

Thus, in another embodiment, the method of the invention comprises the steps of:
i. extracting the cell free nucleic acids from a sample obtained from the subject;
ii. determining the level of a first double stranded DNA fragment having a length inferior to 158 to 170 bp (i.e. short size double stranded DNA fragment);
iii. determining the level of a second double stranded DNA fragment having a length superior or equal to 158 to 170 bp (i.e long size double stranded DNA fragment);
iv. calculating the ratio of the level determined at step ii) to the level determined at step iii) or alternatively the ratio of the level determined at step iii) to the level determined at step ii;
v. comparing the ratio determined at step iv) with a predetermined corresponding reference value and;
vi. concluding that the subject suffers from a cancer when the ratio determined at step iv) differ from the predetermined corresponding reference value.

Thus, in another embodiment, the short size double stranded DNA fragment and the long size double stranded DNA fragment can be determined according to the peak as defined above.

As used herein, the term “ratio of the level” denote the ratio between the levels of the double stranded DNA fragments determined in the steps of the method. According to the invention, the ratio may be the ratio between the level of the first double stranded DNA fragment and the level of the second double stranded DNA fragment (level of the first double stranded DNA fragment/level of the second double stranded DNA fragment) or the ratio between the level of the second double stranded DNA fragment and the level of the first double stranded DNA fragment (level of the second double stranded DNA fragment/level of the first double stranded DNA fragment).

Another aspect of the invention relates to a method for screening a subject for a cancer comprising the steps of:
i. extracting the cell free nucleic acids from a sample obtained from the subject;
ii. determining the level of a group of double stranded DNA fragments on a specific range having a length between 20 to 440 bp;
iii. comparing the level determined at step ii) with a predetermined reference value and;
iv. concluding that the subject suffers from a cancer when the level determined at step ii) differ from the predetermined reference value.

In some embodiment, the level of the group of double stranded DNA fragments determined at step ii) have a length between 20 to 400 base pairs.

Thus, the invention relates to a method for screening a subject for a cancer comprising the steps of:
i. extracting the cell free nucleic acids from a sample obtained from the subject;
ii. determining the level of a group of double stranded DNA fragments on a specific range having a length between 20 to 400 bp;
iii. comparing the level determined at step ii) with a predetermined reference value and;
iv. concluding that the subject suffers from a cancer when the level determined at step ii) differ from the predetermined reference value.

As used herein, the terms “a group of double stranded DNA fragments on a specific rang” denote specific double stranded fragments (more than one fragments) of DNA of different size of nucleic acids whatever the sequence of the fragments. As used herein, the terms “a group of double stranded DNA fragments on a specific range having a length between 20 to 440 bp” denotes specific double stranded fragments (more than one fragments) of DNA of nucleic acids comprises between 20 and 440 bp. It means that between the size of 20 to 400 bp, more than one group of fragments can be selected and their level can be determined. For example, the level of the fragments of 22 bp, the level of the fragments of 57 bp and the level of the fragments of 156 bp can be determined and compared to the a predetermined reference value. As used herein, the term “group” denotes that there is more than one double stranded DNA fragment and at least two double stranded DNA fragments.

According to this method, the level (as defined above) of the fragments of a group of (e.g. composed by) 2, 3, 4, 5, 6, 7, 8, 9, 10, 11, 12, 13, 14, 15, 16, 17, 18, 19, 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; 110; 111; 112; 113; 114; 115; 116; 117; 118; 119; 120; 121; 122; 123; 124; 125; 126; 127; 128; 129; 130; 131; 132; 133; 134; 135; 136; 137; 138; 139; 140; 141; 142; 143; 144; 145, 146; 147; 148; 149; 150; 151; 152; 153; 154; 155; 156; 157; 158; 159; 160; 161; 162; 163; 164; 165; 166; 167; 168; 169; 170; 171; 172; 173; 174; 175; 176; 177; 178; 179; 180; 181; 182; 183; 184; 185; 186; 187; 188; 189; 190; 191; 192; 193; 194; 195; 196; 197; 198; 199; 200; 201; 202; 203; 204; 205; 206; 207; 208; 209; 210; 211; 212; 213; 214; 215; 216; 217; 218; 219; 220; 221; 222; 223; 224; 225; 226; 227; 228; 229; 230; 231; 232; 233; 234; 235; 236; 237; 238; 239; 240; 241; 242; 243; 244; 245; 246; 247; 248, 249; 250; 251; 252; 253; 256; 257; 258; 259; 260; 261; 262; 263; 264; 265; 266; 267; 268; 269; 270; 271; 272; 273; 274; 275; 276; 277; 278; 279; 281; 282; 283; 284; 285; 286; 287; 288; 289; 290; 291; 292; 293; 294; 295; 296; 297; 298; 299; 300; 301; 302; 303; 304; 305; 306; 307; 308; 309; 310; 311; 312; 313; 314; 315; 316; 317; 318; 319; 320; 321; 322; 323; 324; 325; 326; 327; 328; 329; 330; 331; 332; 333; 334; 335; 336; 337; 338; 339; 340; 341; 342; 343; 344; 345; 346; 347; 348; 349; 350; 351; 352; 353; 354; 355; 356; 357; 358; 359; 360; 361; 362; 363; 364; 365; 366; 367; 368; 369; 370; 371; 372; 373; 374; 375; 376; 377; 378; 379, 380, 381, 382, 383, 384, 385, 386, 387, 388, 389, 390, 400, 401, 402, 403, 404, 405, 406, 407, 408, 409, 410, 411, 412, 413, 414, 415, 416, 417, 418, 419 or 420 fragments can be determined.

In one embodiment, the level of a group of double stranded DNA fragments can be determined in specific sub-group and particularly between 20 to 80 bp, between 80 to 155 bp and between 155 to 220 bp whatever the sequences of the fragments. For example, the level of a group of double stranded DNA fragments can be determined in the sub-group of bp having a length between 20 to 80 bp. In this case, for example, the level of bp having a length of 22 bp, 45 bp and 66 bp can be determined.

Particularly, the level of a group of double stranded DNA fragments can be determined between 40 to 145 bp, 170 to 370 bp, 119 to 120 bp, 145 to 250 bp, 30 to 120 bp, 145 to 250 bp or 70 to 150 bp.

In one embodiment, the level of a group of double stranded DNA fragments can be determined in specific sub-group and particularly between 40 to 160 bp, between 40 to 120 bp, between 40 to 80 bp. between 80 to 120 bp, between 130 to 160 bp, between 30 to 220 and between 30 to 145 bp whatever the sequences of the fragments.

In one embodiment, the level of a group of double stranded DNA fragments can be determined in specific sub-group and particularly between 40 to 145 bp, between 40 to 200 bp, between 145 to 250 bp, between 254 to 255 bp, between 145 to 167 bp, between 165 to 250 bp, between 194 to 370 bp and between 119 to 120 bp whatever the sequences of the fragments. For example, the level of a group of double stranded DNA fragments can be determined in the sub-group of bp having a length between 40 to 120 bp. In this case, for example, the level of bp having a length of 42 bp, 65 bp and 96 bp can be determined.

In another embodiment, the invention relates to a method for screening a subject for a cancer comprising the steps of:
i. extracting the cell free nucleic acids from a sample obtained from the subject;
ii. determining the level of a first group of double stranded DNA fragments on a specific range having a length between 20 to 440 bp;
iii. determining the level of a second group of double stranded DNA fragments on a specific rang having a length between 20 to 440 bp;
iv. calculating the ratio of the level determined at step ii) to the level determined at step iii) or alternatively the ratio of the level determined at step iii) to the level determined at step ii;
v. comparing the ratio determined at step iv) with a predetermined corresponding reference value and;
vi. concluding that the subject suffers from a cancer when the ratio determined at step iv) differ from the predetermined corresponding reference value.

In some embodiment, the level of the first group of double stranded DNA fragments and the level of the second group of double stranded DNA fragments can be determined with double stranded DNA fragment on a specific range having a length between 20 to 400 base pairs.

Thus, in some embodiment, the invention relates to a method for screening a subject for a cancer comprising the steps of:
i. extracting the cell free nucleic acids from a sample obtained from the subject;
ii. determining the level of at least one double stranded DNA fragment having a length between 20 to 400 bp;
iii. determining the level of a group of double stranded DNA fragments on a specific rang having a length between 20 to 400 bp;
iv. calculating the ratio of the level determined at step ii) to the level determined at step iii) or alternatively the ratio of the level determined at step iii) to the level determined at step ii;
v. comparing the ratio determined at step iv) with a predetermined corresponding reference value and;
vi. concluding that the subject suffers from a cancer when the ratio determined at step iv) differ from the predetermined corresponding reference value.

In another embodiment, the invention relates to a method for screening a subject for a cancer comprising the steps of:
i. extracting the cell free nucleic acids from a sample obtained from the subject;
ii. determining the level of a first group of double stranded DNA fragments on a specific range having a length between 20 to 440 bp;
iii. determining the level of a second group of double stranded DNA fragments on a specific rang having a length between 20 to 440 bp;
iv. calculating the difference of the level determined at step ii) to the level determined at step iii) or alternatively the ratio of the level determined at step iii) to the level determined at step ii;
v. comparing the difference determined at step iv) with a predetermined corresponding reference value and;
vi. concluding that the subject suffers from a cancer when the difference determined at step iv) differ from the predetermined corresponding reference value.

In another embodiment, the invention relates to a method for screening a subject for a cancer comprising the steps of:
i. extracting the cell free nucleic acids from a sample obtained from the subject;
ii. determining the level of a first group of double stranded DNA fragments on a specific range having a length between 20 to 400 bp;
iii. determining the level of a second group of double stranded DNA fragments on a specific rang having a length between 20 to 400 bp;
iv. calculating the difference of the level determined at step ii) to the level determined at step iii) or alternatively the ratio of the level determined at step iii) to the level determined at step ii;
v. comparing the difference determined at step iv) with a predetermined corresponding reference value and;
vi. concluding that the subject suffers from a cancer when the difference determined at step iv) differ from the predetermined corresponding reference value.

In another embodiment, the invention relates to a method for screening a subject for a cancer comprising the steps of:
i. extracting the cell free nucleic acids from a sample obtained from the subject;
ii. determining the level of a group of double stranded DNA fragments on a specific rang having a length inferior to the peak (i.e. short size double stranded DNA fragment);
iii. determining the level of a group of double stranded DNA fragments on a specific rang having a length superior or equal to the peak (i.e. long double stranded DNA fragment);
iv. calculating the ratio of the level determined at step ii) to the level determined at step iii) or alternatively the ratio of the level determined at step iii) to the level determined at step ii;
v. comparing the ratio determined at step iv) with a predetermined corresponding reference value and;
vi. concluding that the subject suffers from a cancer when the ratio determined at step iv) differ from the predetermined corresponding reference value.

As used herein, the term the “ratio of the level” denote the ratio between the levels of the group of double stranded DNA fragments determined in the steps of the method. According to the invention, the ratio may be the ratio between the level of the first group of double stranded DNA fragment and the level of the second group of double stranded DNA fragment (level of the first group of double stranded DNA fragment/level of second group of double stranded DNA fragment) or the ratio between the level of the second group of double stranded DNA fragment and the level of the first group of double stranded DNA fragment (level of the second group of double stranded DNA fragment/level of the first group of double stranded DNA fragment).

As used herein, the term “a group of double stranded DNA fragments on a specific rang having a length inferior to the peak” denotes a group of double stranded DNA fragments having a length inferior to the peak and superior or equal to 20 bp.

As used herein, the term “a group of double stranded DNA fragments on a specific rang having a length superior or equal to the peak” denotes a group of double stranded DNA fragment having a length superior to the peak and inferior or equal to 440 bp, and more particularly to 400 bp.

As explained below, the peak (as used in the examples) that is to say the value of the length of fragments where the curve of the cancerous subjects and the curve of healthy subject are crossing can correspond to fragments having a length of 158, 159, 160, 161, 162, 163, 164, 165, 166, 167, 168, 169 or 170 bp. These fragments (fragments having a length of 160, 161, 162, 163, 164, 165, 166, 167 or 168 bp) are also the most present (with the maximum level).

Thus, in another embodiment, the invention relates to a method for screening a subject for a cancer comprising the steps of:
i. extracting the cell free nucleic acids from a sample obtained from the subject;
ii. determining the level of a group of double stranded DNA fragments on a specific rang having a length inferior to 158 to 170 bp (i.e. short size double stranded DNA fragment);
iii. determining the level of a group of double stranded DNA fragments on a specific rang having a length superior or equal to 158 to 170 bp (i.e. long double stranded DNA fragment);
iv. calculating the ratio of the level determined at step ii) to the level determined at step iii) or alternatively the ratio of the level determined at step iii) to the level determined at step ii;
v. comparing the ratio determined at step iv) with a predetermined corresponding reference value and;
vi. concluding that the subject suffers from a cancer when the ratio determined at step iv) differ from the predetermined corresponding reference value.

In another aspect of the invention, the ratio between the level of a group of double stranded DNA fragments on a specific range having a length between 20 to 440 bp and the level of one double stranded DNA fragment having a length between 20 to 440 bp can also be determined and compare to a predetermined reference value.

Thus in another aspect, the invention relates a method for screening a subject for a cancer comprising the steps of:
i. extracting the cell free nucleic acids from a sample obtained from the subject;
ii. determining the level of a group of double stranded DNA fragments on a specific range having a length between 20 to 440 bp;
iii. determining the level of one double stranded DNA fragment having a length between 20 to 440 bp;
iv. calculating the ratio of the level determined at step ii) to the level determined at step iii) or alternatively the ratio of the level determined at step iii) to the level determined at step ii;
v. comparing the ratio determined at step iv) with a predetermined corresponding reference value and;
vi. concluding that the subject suffers from a cancer when the ratio determined at step iv) differ from the predetermined corresponding reference value.

In some embodiment, the level of a group of double stranded DNA fragments determined at step ii) have a length between 20 to 400 bp and the level of one double stranded DNA fragment determined at step iii) have a length between 20 to 400 bp.

Thus, the invention relates a method for screening a subject for a cancer comprising the steps of:
i. extracting the cell free nucleic acids from a sample obtained from the subject;
ii. determining the level of a group of double stranded DNA fragments on a specific range having a length between 20 to 400 bp;
iii. determining the level of one double stranded DNA fragment having a length between 20 to 400 bp;
iv. calculating the ratio of the level determined at step ii) to the level determined at step iii) or alternatively the ratio of the level determined at step iii) to the level determined at step ii;
v. comparing the ratio determined at step iv) with a predetermined corresponding reference value and;
vi. concluding that the subject suffers from a cancer when the ratio determined at step iv) differ from the predetermined corresponding reference value.

In one embodiment, the double stranded DNA fragments may have a length inferior to the peak (i.e. short size double stranded DNA fragment) or may have a length superior or equal to the peak (i.e. long size double stranded DNA fragment).

According to the methods of the invention, the double stranded DNA fragment can have a length of 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; 110; 111; 112; 113; 114; 115; 116; 117; 118; 119; 120; 121; 122; 123; 124; 125; 126; 127; 128; 129; 130; 131; 132; 133; 134; 135; 136; 137; 138; 139; 140; 141; 142; 143; 144; 145, 146; 147; 148; 149; 150; 151; 152; 153; 154; 155; 156; 157; 158; 159; 160; 161; 162; 163; 164; 165; 166; 167; 168; 169; 170; 171; 172; 173; 174; 175; 176; 177; 178; 179; 180; 181; 182; 183; 184; 185; 186; 187; 188; 189; 190; 191; 192; 193; 194; 195; 196; 197; 198; 199; 200; 201; 202; 203; 204; 205; 206; 207; 208; 209; 210; 211; 212; 213; 214; 215; 216; 217; 218; 219; 220; 221; 222; 223; 224; 225; 226; 227; 228; 229; 230; 231; 232; 233; 234; 235; 236; 237; 238; 239; 240; 241; 242; 243; 244; 245; 246; 247; 248, 249; 250; 251; 252; 253; 256; 257; 258; 259; 260; 261; 262; 263; 264; 265; 266; 267; 268; 269; 270; 271; 272; 273; 274; 275; 276; 277; 278; 279; 280; 281; 282; 283; 284; 285; 286; 287; 288; 289; 290; 291; 292; 293; 294; 295; 296; 297; 298; 299; 300; 301; 302; 303; 304; 305; 306; 307; 308; 309; 310; 311; 312; 313; 314; 315; 316; 317; 318; 319; 320; 321; 322; 323; 324; 325; 326; 327; 328; 329; 330; 331; 332; 333; 334; 335; 336; 337; 338; 339; 340; 341; 342; 343; 344; 345; 346; 347; 348; 349; 350; 351; 352; 353; 354; 355; 356; 357; 358; 359; 360; 361; 362; 363; 364; 365; 366; 367; 368; 369; 370; 371; 372; 373; 374; 375; 376; 377; 378; 379; 380; 381; 382; 383; 384; 385; 386; 387; 388; 389; 390; 391; 392; 393; 394; 395; 396; 397; 398; 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439 or 440 bp.

According to the methods of the invention, specific group of double stranded DNA fragments can be a group of fragments having a length inferior or superior to 160 bp (or inferior or superior to the peak as defined above), a group of fragments having a length between 165 and 250 bp, between 145 and 165 bp, between 30 and 120 bp, between 100 and 145 bp; between 165 and 250 bp, between 145 and 250, and 300 and 350 bp.

In addition to describe means to discriminate healthy and cancer by determining number or amount of a specific fragment, specific range, and difference or ratio of and comparing them with a reference value as determined here. The inventors describe other means by determining the curve of the difference in cumulative size frequency (ΔS). This curve represent the gain or loss of fragment in function of their size. ΔS is obtained first by calculating the cumulative size value of the size profile of the plasma DNA to test as well as that of reference (or normal or healthy mean) value or mean. Since cancer cfDNA possess higher number of fragments, the cumulative size frequency value of the reference is subtracted from that of plasma DNA to test. The value obtained is the difference of the cumulative size frequency termed ΔS. Plotting ΔS for each DNA length provides a curve from which positive ΔS values corresponds to gain of fragments and negative ΔS values to loss of fragments; the sum of the gain and the loss within all lengths or a range of DNA fragments corresponding to the global gain. As shown in Example 8, plotting the difference of the cumulative size frequency (ΔS values) with using reference values for healthy, enables to discriminate cancer vs healthy plasma by various ways by comparing to healthy individuals: (i), the size at the maximal ΔS value; (ii), the maximal ΔS value; and (iii), the ΔS gain and the ΔS loss. The inventors found that: (1), ΔS gain greatly increased with the % of malignant cell derived cfDNA (up to 12%); 1% being considered as a threshold for screening cancer; and (2), A S global gain greatly increased with the % of malignant cell derived cfDNA (up to 11%); 1% being considered as a threshold for screening cancer. ΔS loss is for all cancer plasma inferior to 1% (Example 8).

Thus, the invention also relates to a method for screening a subject for a cancer comprising the steps of:
i. extracting the cell free nucleic acids (cfDNA) from a sample obtained from the subject;
ii. determining the level of at least one double stranded DNA fragments having a length between 20 to 440 bp;
iii. plotting the cumulative size frequency from the cumulative size frequency reference value values and determining the gain and the loss of the level of cfDNA fragments;
iv. comparing the gain and the loss determined at step iii) with a predetermined reference value and;
v. concluding that the subject suffers from a cancer when the gain and the loss determined at step iii) differ from the predetermined reference value.

In particular embodiment, a ΔS gain and ΔS global gain higher than 1%, in particular higher than 2% is considered as a threshold for screening cancer.

In some embodiment, the level of at least one double stranded DNA fragments determined at step ii) have a length between 20 to 400 bp.

Thus, the invention also relates to a method for screening a subject for a cancer comprising the steps of:
i. extracting the cell free nucleic acids (cfDNA) from a sample obtained from the subject;
ii. determining the level of at least one double stranded DNA fragments having a length between 20 to 400 bp;
iii. plotting the cumulative size frequency from the cumulative size frequency reference value values and determining the gain and the loss of the level of cfDNA fragments;
iv. comparing the gain and the loss determined at step iii) with a predetermined reference value and;
v. concluding that the subject suffers from a cancer when the gain and the loss determined at step iii) differ from the predetermined reference value.

In one embodiment, a step of communicating the result to the patient may be added to all the methods of the invention.

### Comparison Between Single and Double Stranded DNA Fragments

The inventors showed, when they compare specific double stranded DNA fragments and specific single stranded DNA fragments, some differences in the size profile obtained from double stranded DNA fragments and single stranded DNA fragments for each individual (cancerous and healthy patients). The use of the comparison between the size profiles obtained from specific double stranded DNA fragments and specific single stranded DNA fragments (obtained after denaturation) can be thus helpful for screening patients with a cancer. Note, double-stranded DNA unit is base pairs (bp) while single-stranded DNA is nucleotides (nt).

Thus, another aspect of the invention relates to a method for screening a subject for a cancer comprising the steps of:
i. extracting the cell free nucleic acids (cfDNA) from a sample obtained from the subject;
ii. dividing the sample in 2 samples s1 and s2;
iii. determining the level of at least one double stranded DNA fragment having a length between 20 to 440 bp from the sample s1;
iv. denaturating the cell free nucleic acids (cf DNA) of sample s2 to obtain single stranded DNA fragments and determining the level of at least one single stranded DNA fragment having a length between 20 to 440 nucleotides (nt) from said sample s2;
v. calculating the ratio of the level determined at step iii) to the level determined at step iv) or alternatively the ratio of the level determined at step iv) to the level determined at step iii;
vi. comparing the ratio determined at step v) with a predetermined corresponding reference value and;
vii. concluding that the subject suffers from a cancer when the ratio determined at step v) differ from the predetermined corresponding reference value.

In some embodiment, the level of at least one double stranded DNA fragment determined in step iii) from the sample s1 have a length between 20 to 400 bp and the level of at least one single stranded DNA fragment determined at step iv) from the sample s2 have a length between 20 to 400 nt.

Thus, the invention relates to a method for screening a subject for a cancer comprising the steps of:
i. extracting the cell free nucleic acids (cfDNA) from a sample obtained from the subject;
ii. dividing the sample in 2 samples s1 and s2;
iii. determining the level of at least one double stranded DNA fragment having a length between 20 to 400 bp from the sample s1;
iv. denaturating the cell free nucleic acids (cf DNA) of sample s2 to obtain single stranded DNA fragments and determining the level of at least one single stranded DNA fragment having a length between 20 to 400 nucleotides (nt) from said sample s2;
v. calculating the ratio of the level determined at step iii) to the level determined at step iv) or alternatively the ratio of the level determined at step iv) to the level determined at step iii;
vi. comparing the ratio determined at step v) with a predetermined corresponding reference value and;
vii. concluding that the subject suffers from a cancer when the ratio determined at step v) differ from the predetermined corresponding reference value.

Thus, another aspect of the invention relates to a method for screening a subject for a cancer comprising the steps of:
i. extracting the cell free nucleic acids (cfDNA) from a sample obtained from the subject;
ii. dividing the sample in 2 samples s1 and s2;
iii. determining the level of at least one double stranded DNA fragment having a length between 20 to 440 bp from the sample s1;
iv. denaturating the cell free nucleic acids (cf DNA) of sample s2 to obtain single stranded DNA fragments and determining the level of at least one single stranded DNA fragment having a length between 20 to 440 nt from said sample;
v. calculating the difference of the level determined at step iii) to the level determined at step iv) or alternatively the ratio of the level determined at step iv) to the level determined at step iii;
vi. comparing the difference determined at step v) with a predetermined corresponding reference value and;
vii. concluding that the subject suffers from a cancer when the difference determined at step
v) differ from the predetermined corresponding reference value.

In some embodiment, the invention relates to a method for screening a subject for a cancer comprising the steps of:
i. extracting the cell free nucleic acids (cfDNA) from a sample obtained from the subject;
ii. dividing the sample in 2 samples s1 and s2;
iii. determining the level of at least one double stranded DNA fragment having a length between 20 to 400 bp from the sample s1;
iv. denaturating the cell free nucleic acids (cf DNA) of sample s2 to obtain single stranded DNA fragments and determining the level of at least one single stranded DNA fragment having a length between 20 to 400 nt from said sample;
v. calculating the difference of the level determined at step iii) to the level determined at step iv) or alternatively the ratio of the level determined at step iv) to the level determined at step iii;
vi. comparing the difference determined at step v) with a predetermined corresponding reference value and;
vii. concluding that the subject suffers from a cancer when the difference determined at step
v) differ from the predetermined corresponding reference value.

According to the invention, the length of the double or single stranded DNA use to calculate the difference can be calculate between fragments having a length of 110 and 80, 100 and 90, 155 and 125, 90 and 155, 130 and 160, 120 and 150 or 120 and 150 by or nt.

Thus, another aspect of the invention relates to a method for screening a subject for a cancer comprising the steps of:
i. extracting the cell free nucleic acids (cfDNA) from a sample obtained from the subject;
ii. dividing the sample in 2 samples s1 and s2;
iii. determining the level of a group of double of at least one double stranded DNA fragment having a length between 20 to 440 bp from the sample s1;
iv. denaturating the cell free nucleic acids (cf DNA) of sample s2 to obtain single stranded DNA fragments and determining the level of a group of at least one single stranded DNA fragment having a length between 20 to 440 nt from said sample s2;
v. calculating the ratio of the level determined at step iii) to the level determined at step
iv) or alternatively the ratio of the level determined at step iv) to the level determined at step iii;
vi. comparing the ratio determined at step v) with a predetermined corresponding reference value and;
vii. concluding that the subject suffers from a cancer when the ratio determined at step v) differ from the predetermined corresponding reference value.
   In some embodiment, the level of the group of double at least one double stranded DNA fragment determined in step iii) from the sample s1 have a length between 20 to 400 bp, and the level of the group of at least one single stranded DNA fragment determined at step iv) from the sample s2 have a length between 20 to 400 bp.

Thus, the invention relates to a method for screening a subject for a cancer comprising the steps of:
i. extracting the cell free nucleic acids (cfDNA) from a sample obtained from the subject;
ii. dividing the sample in 2 samples s1 and s2;
iii. determining the level of a group of double of at least one double stranded DNA fragment having a length between 20 to 400 bp from the sample s1;
iv. denaturating the cell free nucleic acids (cf DNA) of sample s2 to obtain single stranded DNA fragments and determining the level of a group of at least one single stranded DNA fragment having a length between 20 to 400 nt from said sample s2;
v. calculating the ratio of the level determined at step iii) to the level determined at step
iv) or alternatively the ratio of the level determined at step iv) to the level determined at step iii;
vi. comparing the ratio determined at step v) with a predetermined corresponding reference value and;
vii. concluding that the subject suffers from a cancer when the ratio determined at step v) differ from the predetermined corresponding reference value.

Particularly, the level of a group of double stranded DNA fragments or single stranded DNA fragment can be determined between 70 to 150 bp/nt, 70 to 145 bp/nt or 40 to 145 bp/nt. Particularly, the ratio between two group can be done and particularly, between 120 to 145/194 to 370, between 119 to 120/194 to 370 or 119 to 120/254 to 255 bp/nt.

Thus, another aspect of the invention relates to a method for screening a subject for a cancer comprising the steps of:
i. extracting the cell free nucleic acids (cfDNA) from a sample obtained from the subject;
ii. dividing the sample in 2 samples s1 and s2;
iii. determining the level of a group of double of at least one double stranded DNA fragment having a length between 20 to 440 bp from the sample s1;
iv. denaturating the cell free nucleic acids (cf DNA) of sample s2 to obtain single stranded DNA fragments and determining the level of a group of at least one single stranded DNA fragment having a length between 20 to 440 nt from said sample s2;
v. calculating the difference of the level determined at step iii) to the level determined at step iv) or alternatively the ratio of the level determined at step iv) to the level determined at step iii;
vi. comparing the difference determined at step v) with a predetermined corresponding reference value and;
vii. concluding that the subject suffers from a cancer when the difference determined at step v) differ from the predetermined corresponding reference value.

Thus, another aspect of the invention relates to a method for screening a subject for a cancer comprising the steps of:
i. extracting the cell free nucleic acids (cfDNA) from a sample obtained from the subject;
ii. dividing the sample in 2 samples s1 and s2;
iii. determining the level of a group of double of at least one double stranded DNA fragment having a length between 20 to 400 bp from the sample s1;
iv. denaturating the cell free nucleic acids (cf DNA) of sample s2 to obtain single stranded DNA fragments and determining the level of a group of at least one single stranded DNA fragment having a length between 20 to 400 nt from said sample s2;
v. calculating the difference of the level determined at step iii) to the level determined at step iv) or alternatively the ratio of the level determined at step iv) to the level determined at step iii;
vi. comparing the difference determined at step v) with a predetermined corresponding reference value and;
vii. concluding that the subject suffers from a cancer when the difference determined at step v) differ from the predetermined corresponding reference value.

According to the methods of the invention, the double stranded DNA fragment can have a length of 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; 110; 111; 112; 113; 114; 115; 116; 117; 118; 119; 120; 121; 122; 123; 124; 125; 126; 127; 128; 129; 130; 131; 132; 133; 134; 135; 136; 137; 138; 139; 140; 141; 142; 143; 144; 145, 146; 147; 148; 149; 150; 151; 152; 153; 154; 155; 156; 157; 158; 159; 160; 161; 162; 163; 164; 165; 166; 167; 168; 169; 170; 171; 172; 173; 174; 175; 176; 177; 178; 179; 180; 181; 182; 183; 184; 185; 186; 187; 188; 189; 190; 191; 192; 193; 194; 195; 196; 197; 198; 199; 200; 201; 202; 203; 204; 205; 206; 207; 208; 209; 210; 211; 212; 213; 214; 215; 216; 217; 218; 219; 220; 221; 222; 223; 224; 225; 226; 227; 228; 229; 230; 231; 232; 233; 234; 235; 236; 237; 238; 239; 240; 241; 242; 243; 244; 245; 246; 247; 248, 249; 250; 251; 252; 253; 256; 257; 258; 259; 260; 261; 262; 263; 264; 265; 266; 267; 268; 269; 270; 271; 272; 273; 274; 275; 276; 277; 278; 279; 280; 281; 282; 283; 284; 285; 286; 287; 288; 289; 290; 291; 292; 293; 294; 295; 296; 297; 298; 299; 300; 301; 302; 303; 304; 305; 306; 307; 308; 309; 310; 311; 312; 313; 314; 315; 316; 317; 318; 319; 320; 321; 322; 323; 324; 325; 326; 327; 328; 329; 330; 331; 332; 333; 334; 335; 336; 337; 338; 339; 340; 341; 342; 343; 344; 345; 346; 347; 348; 349; 350; 351; 352; 353; 354; 355; 356; 357; 358; 359; 360; 361; 362; 363; 364; 365; 366; 367; 368; 369; 370; 371; 372; 373; 374; 375; 376; 377; 378; 379; 380; 381; 382; 383; 384; 385; 386; 387; 388; 389; 390; 391; 392; 393; 394; 395; 396; 397; 398; 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439 or 440 nucleotides.

As explained above, the inventors describe other means to discriminate healthy and cancer by determining the curve of the difference in cumulative size frequency (ΔS).

Thus, the invention also relates to a method for screening a subject for a cancer comprising the steps of:
i. extracting the cell free nucleic acids (cfDNA) from a sample obtained from the subject;
ii. dividing the sample in 2 samples s1 and s2;
iii. determining the level of double stranded DNA fragment having a length between 20 to 440 bp from the sample s1;
iv. denaturating the cell free nucleic acids (cf DNA) of sample s2 to obtain single stranded DNA fragments and determining the level of single stranded DNA fragment having a length between 20 to 440 nt from said sample s2;
v. plotting the cumulative size frequency obtained from s1 and s2 from the cumulative size frequency reference values;
vi. plotting the difference of the ΔS values from s1 and s2 obtained in step v) and determining the gain and the loss of fragments;
vii. comparing the gain and the loss determined at step vi) with a predetermined reference value and;
viii. concluding that the subject suffers from a cancer when the gain or the loss determined at step vi) differ from the predetermined reference value.

In some embodiment, the level of double stranded DNA fragment determined at step iii) have a length between 20 to 400 bp, and the level of single stranded DNA fragment determined at step iv) have a length between 20 to 400 nt.

Thus, the invention also relates to a method for screening a subject for a cancer comprising the steps of:
i. extracting the cell free nucleic acids (cfDNA) from a sample obtained from the subject;
ii. dividing the sample in 2 samples s1 and s2;
iii. determining the level of double stranded DNA fragment having a length between 20 to 400 bp from the sample s1;
iv. denaturating the cell free nucleic acids (cf DNA) of sample s2 to obtain single stranded DNA fragments and determining the level of single stranded DNA fragment having a length between 20 to 400 nt from said sample s2;
v. plotting the cumulative size frequency obtained from s1 and s2 from the cumulative size frequency reference values;
vi. plotting the difference of the ΔS values from s1 and s2 obtained in step v) and determining the gain and the loss of fragments;
vii. comparing the gain and the loss determined at step vi) with a predetermined reference value and;
viii. concluding that the subject suffers from a cancer when the gain or the loss determined at step vi) differ from the predetermined reference value.

In a particular embodiment, a ΔS gain and ΔS global gain higher than 11%, and 5% respectively being considered as a threshold for screening cancer.

### Analyse of Double and Simple Stranded DNA Fragments Per Chromosome

The inventors also showed an higher fragmentation in several chromosomes which can allow to discriminate cancer plasma with healthy plasma by examining fragmentation level per chromosome.

Accordingly, detection of the “natural” double-stranded DNA fragments or of the artificial single-stranded DNA fragments of the invention can allow a discrimination between all the chromosomes in respect to cancer or healthy status. Particularly, fragments derived from the chromosomes 1, 2, 4, 5, 6, 8, 9, 10, 11, 13, 18, 20, X or Y can be used to discriminate cancer or healthy subjects. Particularly, fragments derived from the chromosomes 2, 6, 8, 10, 11, 20 or Y can be used to discriminate cancer or healthy subjects.

### Methods Used to Determine the Lengths of Fragments According to the Invention

Methods to determine the length of simple or double stranded DNA fragment may be accomplished by any method, including without limitation chromatography, direct sequencing, spectrometry or Q-PCR.

Direct sequencing may be accomplished by any method, including without limitation chemical sequencing using the Maxam-Gilbert method (12, 13), spectrometry and particularly mass spectrometry sequencing and sequencing using a chip-based technology.

In the chemical sequencing, base specific modifications result in a base specific cleavage of the radioactive or fluorescently labeled DNA fragment. With the four separate base specific cleavage reactions, four sets of nested fragments are produced which are separated according to length by polyacrylamide gel electrophoresis (PAGE). After autoradiography, the sequence can be read directly since each band (fragment) in the gel originates from a base specific cleavage event. Thus, the fragment lengths in the four “ladders” directly translate into a specific position in the DNA sequence.

In the enzymatic sequencing, the four base specific sets of DNA fragments are formed by starting with a primer/template system elongating the primer into the unknown DNA sequence area and thereby copying the template and synthesizing a complementary strand by DNA polymerases, such as Klenow fragment of E. coli DNA polymerase I, a DNA polymerase from Thermus aquaticus, Taq DNA polymerase, or a modified T7 DNA polymerase, Sequenase(14), in the presence of chain-terminating reagents.

Several new methods for DNA sequencing (High-throughput sequencing (HTS) methods) were developed in the mid to late 1990s and were implemented in commercial DNA sequencers by the year 2000. Together these were called the “next-generation” or “second-generation”sequencing methods. These HTS included but are not limited to: Single-molecule real-time sequencing, Ion semiconductor, Pyrosequencing, Sequencing by synthesis, Sequencing by ligation, Nanopore Sequencing, Chain termination and Sequencing by hybridization. Some of these methods allow a Whole Gene Sequencing (WGS), Whole Exome Sequencing (WES) or a Targeted Sequencing.

Here, the inventors directly compared the cfDNA size profiles of cancer-patient cfDNA extract obtained by SSP-S and DSP-S. SSP-S artificially detect single-stranded DNA whereas no single-stranded cfDNA exist in the human body and their detection results from an initial step of physical denaturation separating both strands of the DNA molecule. Then the unit of the size of single-stranded DNA should be nucleotides (nt). Nevertheless, when comparing DSP-S and SSP-S cfDNA size profile nt can be directly compared to bp since those single-stranded DNA were naturally hybridized with the opposite strand forming a double-stranded DNA fragment intimately associated with the nucleosome in the blood stream.

When directly comparing the cfDNA size profiles of cancer-patient cfDNA extract obtained by SSP-S and DSP-S, a high discrepancy was found. Artificially detected single-stranded DNA fragments are shorter than detected double-stranded DNA fragments essentially because of nicks resulting from DNase 1 attacks on the DNA molecules. The population corresponding to the DNA size wrapped around a mononucleosome (120-220-bp range, peaking up to 165-167 bp) was observed using both methods. Such a peaking value was analogous to that of several reports analyzing cfDNA (11, 18, 19) and the plasma of pregnant women or organ transplant recipients (9), suggesting DNA fragmentation during apoptosis (20, 21). However, SSP-S revealed a substantial cfDNA fragment population ranging from 30 to 100 nt, which was not detectable using the DSP library. CfDNA appeared more accessible for sequencing following SSP-S, as previously reported by our group (10) and then by Burnham et al. (16) Fragments shorter than 100 nt are in abundance in cancer-patient-derived plasma, but conventional DSP-S methods appeared insensitive to ultra-short cfDNA, emphasizing the need to use SSP-S for optimally examining cfDNA profiles. The SSP library has been recently described and used to generate high-resolution genomes when examining paleonthologican ancient DNA (15, 16). This method uses a single strand DNA ligase and a 5′ phosphorylated and biotinylated adapter oligobp to capture and bind single strand DNA molecules to beads without prior end-repair(15). Double stranded DNA is generated through use of primers from this ligation, and subsequently receives a second adaptor via blunt-end ligation. Completion of the adaptor sequence through an amplification reaction is then carried out from finished single strands obtained by heating the previously obtained molecules.

### Predetermined Corresponding Reference Values

Typically, the predetermined corresponding reference value can be relative to a number or value derived from population studies, including without limitation, subjects of the same or similar age range, subjects in the same or similar ethnic group, subjects at risk of cancer, subjects having the same severity of cancer and subject without cancer (healthy subject). Such predetermined corresponding reference values can be derived from statistical analyses and/or risk prediction data of populations obtained from mathematical algorithms and computed indices of the disease.

Typically, the predetermined corresponding reference value is a threshold value or a cut-off value. A “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of the expression level of the marker of interest (e.g. double stranded DNA fragments for a specific length, ratio between single/double stranded DNA fragments for a specific length or between group of single/double stranded DNA fragments for a specific length) in properly banked historical subject samples may be used in establishing the predetermined corresponding reference value. In some embodiments, the predetermined corresponding reference value is the median measured in the population of the subjects for the marker of interest (e.g. double stranded DNA fragments for a specific length, ratio between single/double stranded DNA fragments for a specific length or between group of single/double stranded DNA fragments for a specific length). In some embodiments, the threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the expression level of the marker of interest (e.g. double stranded DNA fragments for a specific length, ratio between single/double stranded DNA fragments for a specific length or between group of single/double stranded DNA fragments for a specific length) in a group of reference, one can use algorithmic analysis for the statistic treatment of the expression levels determined in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator the reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the predetermined corresponding reference value is typically determined by carrying out a method comprising the steps of:

a) providing a collection of samples from subjects;

b) providing, for each sample provided at step a), information relating to the actual clinical profile of the subject (healthy or suffering from a cancer);

c) providing a serial of arbitrary quantification values;

d) determining the level of the marker of interest (e.g. double stranded DNA fragments for a specific length, ratio between single/double stranded DNA fragments for a specific length or between group of single/double stranded DNA fragments for a specific length) for each sample contained in the collection provided at step a);

e) classifying said blood samples in two groups for one specific arbitrary quantification value provided at step c), respectively: (i) a first group comprising samples that exhibit a quantification value for level that is lower than the said arbitrary quantification value contained in the said serial of quantification values; (ii) a second group comprising samples that exhibit a quantification value for said level that is higher than the said arbitrary quantification value contained in the said serial of quantification values; whereby two groups of samples are obtained for the said specific quantification value, wherein the samples of each group are separately enumerated;

f) calculating the statistical significance between (i) the quantification value obtained at step e) and (ii) the actual clinical profile of the subjects from which samples contained in the first and second groups defined at step f) derive;

g) reiterating steps f) and g) until every arbitrary quantification value provided at step d) is tested;

h) setting the said predetermined corresponding reference value as consisting of the arbitrary quantification value for which the highest statistical significance (most significant) has been calculated at step g).

Thus in some embodiments, the predetermined corresponding reference value thus allows discrimination between healthy subject and subjects suffering from cancer. Practically, high statistical significance values (e.g. low P values) are generally obtained for a range of successive arbitrary quantification values, and not only for a single arbitrary quantification value. Thus, in one alternative embodiment of the invention, instead of using a definite predetermined corresponding reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P value) are retained, so that a range of quantification values is provided. This range of quantification values includes a “cut-off” value as described above. For example, according to this specific embodiment of a “cut-off” value, the diagnosis can be determined by comparing the level of the marker of interest (e.g. single stranded DNA fragments for a specific length, ratio between single stranded DNA fragments for a specific length or between group of single stranded DNA fragments for a specific length) with the range of values which are identified. In certain embodiments, a cut-off value thus consists of a range of quantification values, e.g. centered on the quantification value for which the highest statistical significance value is found (e.g. generally the minimum p value which is found). For example, on a hypothetical scale of 1 to 10, if the ideal cut-off value (the value with the highest statistical significance) is 5, a suitable (exemplary) range may be from 4-6. Therefore, a subject may be assessed by comparing values obtained by measuring the level of the marker of interest (e.g. single stranded DNA fragments for a specific length, ratio between single stranded DNA fragments for a specific length or between group of single stranded DNA fragments for a specific length), where values higher (or lower depending on the selected marker) than 5 reveal that the subject suffers from cancer and values lower (or higher depending on the selected marker) than 5 reveal that the subject does not suffer from a cancer. In some embodiments, a subject may be screened for a cancer by comparing values obtained by measuring the level of the marker of interest (e.g. single stranded DNA fragments for a specific length, ratio between single stranded DNA fragments for a specific length or between group of single stranded DNA fragments for a specific length) and comparing the values on a scale, where values above (or below depending on the selected marker) the range of 4-6 indicate that the subject suffers from a cancer and values below or above depending on the selected marker) the range of 4-6 indicate that the subject does not suffer from a cancer, with values falling within the range of 4-6 indicate that further investigation are needed for determining whether the subject suffers from a cancer.

According to the invention, the variation of the level of the double stranded DNA fragments or the level of a group of double stranded DNA fragments may be evaluated.

According to the invention, discriminations are globally determined either from proportion (in relation to total fragments) of a single fragment or a fragments group, or from a ratio of two specific sizes of fragments or of a specific size relative to a group of fragments size or of fragments size group with respect to another fragments size group.

In one embodiment, a “support vector machine (SVM)” can be used to all the methods of the invention for the steps of determination of the level of double or single stranded DNA or for the step of comparison of the level with the predetermined reference values or for any statistical method used in the invention.

As used herein, the term “support vector machine (SVM)” has its general meaning in the art and refers to a universal learning machine useful as a statistical tool for classification and using an algorithm developed by Cortes and Vapnik (Cortes C. and Vapnik V. N. “Support-vector networks” Machine Learning 1995, 20(3):273-297).

### Therapeutic Applications

The methods of the present invention can also be suitable for determining whether a subject is eligible or not to an anti-cancer treatment. An anti-cancer treatment typically consists of radiotherapy, chemotherapy, immunotherapy or a combination thereof. The treatment can also consist of an adjuvant therapy (i.e. treatment after chirurgical resection of the primary tumor) of a neoadjuvant therapy (i.e. treatment before chirurgical resection of the primary tumor).

In some embodiments, the methods of the present invention are suitable for determining whether a subject is eligible or not to a treatment with a chemotherapeutic agent. For example, when it is concluded that the subject has a cancer then the physician can take the choice to administer the subject with a chemotherapeutic agent.

The term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., (22); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and phannaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and phannaceutically acceptable salts, acids or derivatives of any of the above.

In some embodiments, the methods of the present invention are suitable for determining whether a subject is eligible or not to targeted therapy. For example, when it is concluded that the subject has a cancer then the physician can take the choice to administer the subject with a targeted therapy.

Targeted cancer therapies are drugs or other substances that block the growth and spread of cancer by interfering with specific molecules (“molecular targets”) that are involved in the growth, progression, and spread of cancer. Targeted cancer therapies are sometimes called “molecularly targeted drugs,” “molecularly targeted therapies,” “precision medicines,” or similar names.

In some embodiments, the targeted therapy consists of administering the subject with a tyrosine kinase inhibitor. The term “tyrosine kinase inhibitor” refers to any of a variety of therapeutic agents or drugs that act as selective or non-selective inhibitors of receptor and/or non-receptor tyrosine kinases. Tyrosine kinase inhibitors and related compounds are well known in the art and described in U.S Patent Publication 2007/0254295, which is incorporated by reference herein in its entirety. It will be appreciated by one of skill in the art that a compound related to a tyrosine kinase inhibitor will recapitulate the effect of the tyrosine kinase inhibitor, e.g., the related compound will act on a different member of the tyrosine kinase signaling pathway to produce the same effect as would a tyrosine kinase inhibitor of that tyrosine kinase. Examples of tyrosine kinase inhibitors and related compounds suitable for use in methods of embodiments of the present invention include, but are not limited to, dasatinib (BMS-354825), PP2, BEZ235, saracatinib, gefitinib (Iressa), sunitinib (Sutent; SU11248), erlotinib (Tarceva; OSI-1774), lapatinib (GW572016; GW2016), canertinib (CI 1033), semaxinib (SU5416), vatalanib (PTK787/ZK222584), sorafenib (BAY 43-9006), imatinib (Gleevec; STI571), leflunomide (SU101), vandetanib (Zactima; ZD6474), MK-2206 (8-[4-aminocyclobutyl)phenyl]-9-phenyl-1,2,4-triazolo[3,4-f][1,6]naphthyridin-3 (2H)-one hydrochloride) derivatives thereof, analogs thereof, and combinations thereof. Additional tyrosine kinase inhibitors and related compounds suitable for use in the present invention are described in, for example, U.S Patent Publication 2007/0254295, U.S. Pat. Nos. 5,618,829, 5,639,757, 5,728,868, 5,804,396, 6,100,254, 6,127,374, 6,245,759, 6,306,874, 6,313,138, 6,316,444, 6,329,380, 6,344,459, 6,420,382, 6,479,512, 6,498,165, 6,544,988, 6,562,818, 6,586,423, 6,586,424, 6,740,665, 6,794,393, 6,875,767, 6,927,293, and 6,958,340, all of which are incorporated by reference herein in their entirety. In certain embodiments, the tyrosine kinase inhibitor is a small molecule kinase inhibitor that has been orally administered and that has been the subject of at least one Phase I clinical trial, more preferably at least one Phase II clinical, even more preferably at least one Phase III clinical trial, and most preferably approved by the FDA for at least one hematological or oncological indication. Examples of such inhibitors include, but are not limited to, Gefitinib, Erlotinib, Lapatinib, Canertinib, BMS-599626 (AC-480), Neratinib, KRN-633, CEP-11981, Imatinib, Nilotinib, Dasatinib, AZM-475271, CP-724714, TAK-165, Sunitinib, Vatalanib, CP-547632, Vandetanib, Bosutinib, Lestaurtinib, Tandutinib, Midostaurin, Enzastaurin, AEE-788, Pazopanib, Axitinib, Motasenib, OSI-930, Cediranib, KRN-951, Dovitinib, Seliciclib, SNS-032, PD-0332991, MKC-I (Ro-317453; R-440), Sorafenib, ABT-869, Brivanib (BMS-582664), SU-14813, Telatinib, SU-6668, (TSU-68), L-21649, MLN-8054, AEW-541, and PD-0325901.

In some embodiments, the methods of the present invention are suitable for determining whether a subject is eligible or not to a treatment with an immunotherapeutic agent. For example, when it is concluded that the subject has a cancer then the physician can take the choice to administer the subject with an immunotherapeutic agent.

The term “immunotherapeutic agent,” as used herein, refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies and non-cytokine adjuvants. Alternatively the immunotherapeutic treatment may consist of administering the subject with an amount of immune cells (T cells, NK, cells, dendritic cells, B cells . . . ).

Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents.

Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants.

A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors.

Interferons (IFNs) contemplated by the present invention include the common types of IFNs, IFN-alpha (IFN-a), IFN-beta (IFN-beta) and IFN-gamma (IFN-y). IFNs can act directly on cancer cells, for example, by slowing their growth, promoting their development into cells with more normal behaviour and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation). The use of IFN-alpha, alone or in combination with other immunotherapeutics or with chemotherapeutics, has shown efficacy in the treatment of various cancers including melanoma (including metastatic melanoma), renal cancer (including metastatic renal cancer), breast cancer, prostate cancer, and cervical cancer (including metastatic cervical cancer).

Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. Interleukins, alone or in combination with other immunotherapeutics or with chemotherapeutics, have shown efficacy in the treatment of various cancers including renal cancer (including metastatic renal cancer), melanoma (including metastatic melanoma), ovarian cancer (including recurrent ovarian cancer), cervical cancer (including metastatic cervical cancer), breast cancer, colorectal cancer, lung cancer, brain cancer, and prostate cancer.

Interleukins have also shown good activity in combination with IFN-alpha in the treatment of various cancers (23, 24).

Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin). Colony stimulating factors have shown efficacy in the treatment of cancer, including melanoma, colorectal cancer (including metastatic colorectal cancer), and lung cancer.

Non-cytokine adjuvants suitable for use in the combinations of the present invention include, but are not limited to, Levamisole, alum hydroxide (alum), Calmette-Guerin bacillus (ACG), incomplete Freund's Adjuvant (IFA), QS-21, DETOX, Keyhole limpet hemocyanin (KLH) and dinitrophenyl (DNP). Non-cytokine adjuvants in combination with other immuno- and/or chemotherapeutics have demonstrated efficacy against various cancers including, for example, colon cancer and colorectal cancer (Levimasole); melanoma (BCG and QS-21); renal cancer and bladder cancer (BCG).

In addition to having specific or non-specific targets, immunotherapeutic agents can be active, i.e. stimulate the body's own immune response, or they can be passive, i.e. comprise immune system components that were generated external to the body.

Passive specific immunotherapy typically involves the use of one or more monoclonal antibodies that are specific for a particular antigen found on the surface of a cancer cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins.

Monoclonal antibodies currently used as cancer immunotherapeutic agents that are suitable for inclusion in the combinations of the present invention include, but are not limited to, rituximab (Rituxan®), trastuzumab (Herceptin®), ibritumomab tiuxetan (Zevalin®), tositumomab (Bexxar®), cetuximab (C-225, Erbitux®), bevacizumab (Avastin®), gemtuzumab ozogamicin (Mylotarg®), alemtuzumab (Campath®), and BL22. Monoclonal antibodies are used in the treatment of a wide range of cancers including breast cancer (including advanced metastatic breast cancer), colorectal cancer (including advanced and/or metastatic colorectal cancer), ovarian cancer, lung cancer, prostate cancer, cervical cancer, melanoma and brain tumours. Other examples include anti-CTLA4 antibodies (e.g. Ipilimumab), anti-PD1 antibodies, anti-PDL1 antibodies, anti-TIMP3 antibodies, anti-LAG3 antibodies, anti-B7H3 antibodies, anti-B7H4 antibodies or anti-B7H6 antibodies.

Active specific immunotherapy typically involves the use of cancer vaccines. Cancer vaccines have been developed that comprise whole cancer cells, parts of cancer cells or one or more antigens derived from cancer cells. Cancer vaccines, alone or in combination with one or more immuno- or chemotherapeutic agents are being investigated in the treatment of several types of cancer including melanoma, renal cancer, ovarian cancer, breast cancer, colorectal cancer, and lung cancer. Non-specific immunotherapeutics are useful in combination with cancer vaccines in order to enhance the body's immune response.

The immunotherapeutic treatment may consist of an adoptive immunotherapy as described by Nicholas P. Restifo et al.(25), in adoptive immunotherapy, the subject's circulating lymphocytes, or tumor infiltrated lymphocytes, are isolated in vitro, activated by lymphokines such as IL-2 or transuded with genes for tumor necrosis, and readministered(26, 27). The activated lymphocytes are most preferably be the subject's own cells that were earlier isolated from a blood or tumor sample and activated (or “expanded”) in vitro. This form of immunotherapy has produced several cases of regression of melanoma and renal carcinoma.

In some embodiments, the methods of the present invention are suitable for determining whether a subject is eligible or not to a treatment with a radiotherapeutic agent. For example, when it is concluded that the subject has a cancer then the physician can take the choice to administer the subject with a radiotherapeutic agent.

The term “radiotherapeutic agent” as used herein, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy. Such methods can optionally further comprise the administration of one or more additional cancer therapies, such as, but not limited to, chemotherapies, and/or another radiotherapy.

The methods of the present invention are also suitable for determining the efficiency of an above mentioned treatment in the subject.

Thus, the invention also relates to a method for determining whether a subject achieve a response with a treatment comprising the steps of:
i. apply a method according to the invention before the treatment;
ii. apply a method according to the invention after the treatment;
iii. comparing the values determined at step i) with the value determined at step and;
iv. concluding that the subject suffers from a cancer when the level determined at step i) differ from the value determined at step ii).

The above mentioned methods of the present invention are particularly suitable for discriminating responder from non-responder. As used herein the term “responder” in the context of the present disclosure refers to a subject that will achieve a response, i.e. a subject where the cancer is eradicated, reduced or improved, or stabilized such that the disease is not progressing after the treatment. In responders where the cancer is stabilized, the period of stabilization is such that the quality of life and/or subjects' life expectancy is increased (for example stable disease for more than 6, 7, 8, 9, 10, 11, 12, 13, 14, 15, 16, 17, 18, 19, 20, 21, 22, 23, 24 or more months) in comparison to a subject that does not receive the treatment. A non-responder or refractory subject includes subjects for whom the cancer does not show reduction or improvement after treatment. Optionally the characterization of the subject as a responder or non-responder can be performed by reference to a standard or a training set. The standard may be the profile of a subject who is known to be a responder or non responder or alternatively may be a numerical value. Such predetermined standards may be provided in any suitable form, such as a printed list or diagram, computer software program, or other media. When it is concluded that the subject is a non responder, the physician could take the decision to stop the treatment to avoid any further adverse sides effects.

The methods of the present invention are also suitable for determining the efficiency of an above mentioned treatment in the subject.

Thus, the invention also relates to a method for determining whether a subject who suffered from a cancer has a relapse after a treatment comprising the steps of:
i. apply a method according to the invention after the treatment;
ii. comparing the values determined at step i) with a predetermined reference value and;
iii. concluding that the subject has a relapse when the level determined at step i) differ from the predetermined reference value.

As used herein, the term “relapse” refers to the return of a cancer or the signs and symptoms of a cancer after a period of improvement in which no cancer could be detected. The likely relapse occurs is that a few of the original cancer cells survived the initial treatment. Alternatively, this is because cancer cells spread to other parts of the body and were too small to be detected during the follow-up taking place after the treatment (metastasis).

The invention will be further illustrated by the following figures and examples. However, these examples and figures should not be interpreted in any way as limiting the scope of the present invention.

### FIGURES

FIG. 1: Comparison of the cfDNA size profile from healthy and cancer individuals by sequencing from either single or double strand DNA library preparation. A and B, size profiles obtained from four healthy individuals; C and D, size profiles obtained from three cancer individuals (full line) and the mean of four healthy individuals (dotted line). Size profiles obtained following sequencing from either single (B,D) or double strand (A,C) DNA library preparation.

FIG. 2: Illustrative detailed comparison of the size profile of cfDNA from a cancer patient and healthy individuals. #2 cancer patient (full line), mean of four healthy individuals (dotted lines); DSP sequencing (A) and SSP sequencing (B). Insert, zoom on the 240 to 400 nt or bp size range.

FIG. 3: Difference between healthy subjects and individual cancer patient cfDNA size profiles as determined by DSP sequencing. Patient #1 (A, B, C), 2 (D, E, F) and 3 (G, H, I). Size distributions are shown in A, D, G, plot of cumulative size frequencies for cancer DNA (full line), healthy DNA (dotted line) in B, E, H; the difference in cumulative frequencies, denoted as ΔS, between individual cancer and healthy DNA mean are shown in C, F, I.

FIG. 4: Difference between healthy and individual cancer patient cfDNA size profiles as determined by SSP sequencing. Patient #1 (A, B, C), #2 (D, E, F) and #3 (G, H, I). Size distributions are shown in (A, D, G), plot of cumulative size frequencies for cancer DNA (full line), healthy DNA (dotted line) in (B, E, H); the difference in cumulative size frequencies, denoted as ΔS, between individual cancer and healthy DNA mean are shown in C, F, I.

FIG. 5: Difference between individual cancer patient cfDNA size profiles as determined by DSP or SSP sequencing. Patient #1 (A, B, C), 2 (D, E, F) and 3 (G, H, I) and the mean of four healthy individuals (J, K, L). Size distributions are shown in (A, D, G, J), plot of cumulative size frequencies for cancer DNA obtained from SSP (full line) and DSP (dotted line) sequencing in (B, E, H, K); the difference in cumulative size frequencies, denoted as ΔS, between SSP and DSP sequencing are shown in C, F, I, and L.

FIG. 6: Difference in cumulative size frequencies. A, Difference in cumulative frequencies, denoted as ΔS, between SSP (full line) and DSP (dotted line) sequencing for the mean of the three cancer patients. B, Difference in cumulative frequencies in obtained in FIG. 6A, denoted as ΔS, between SSP and DSP sequencing for the mean of the three cancer patients. C, Difference in cumulative frequencies, denoted as ΔS, between SSP and DSP sequencing for each of the three cancer patients (full line) and the mean from the four healthy individual (dotted line).

FIG. 7: Fragmentation level per chromosome following DSP-S(A and B) or SSP-S (C and D) in the plasma of the four healthy individuals (A and C) and in the plasma of the three cancer patients (B and D). Each line represent the values for one individual plasma. Number of reads, per sample, mapping to each chromosome, irrespective of read length. These values are the expected/observed # of reads mapping to each chromosome for a diploid genome. Values are presented for each chromosome respective to chromosome 1. A value of 100% means that particular chromosome is not over/underrepresented for a normal diploid genome. The more reads are detected, the higher is the fragmentation, the more short fragments are detected as compared to the mean of fragments.

TABLE 1
Detailed characterization of the ~10 bp bp/nt periodicity
subpeaks observed from size distribution of cfDNA of cancer patients
as determined by DSP-S (top panel) and SSP-S (bottom panel)

| | Subpeak corresponding size, DSP-S (bp) | | | | | | |
|---|---|---|---|---|---|---|---|
| | Cancer | | | Healthy | | | |
| Peak | 1 DS | 2 DS | 3 DS | 5 DS | 6 DS | 7 DS | 8 DS |
| 1 | — | — | 73 | — | — | — | — |
| 2 | 79 | 81 | 81 | 80 | 81 | 80 | 81 |
| 3 | 91 | 91 | 91 | 91 | 91 | 91 | 92 |
| 4 | 102 | 102 | 102 | 101 | 102 | 101 | 101 |
| 5 | 111 | 112 | 112 | 112 | 111 | 112 | 111 |
| 6 | 121 | 122 | 122 | 122 | 122 | 122 | 122 |
| 7 | 133 | 134 | 133 | 134 | 134 | 134 | 133 |
| 8 | 141 | 142 | 142 | 142 | 143 | 142 | 143 |
| 9 | — | — | — | 146 | 145 | 146 | 145 |
| 10 | 149 | 152 | 149 | 152 | 152 | 152 | 152 |
| 11 | 164 | 166 | 166 | 166 | 167 | 166 | 167 |
| 12 | 252 | — | 272 | — | — | — | — |
| | Subpeak corresponding size, SSP-S (nt) | | | | | | |
| | Cancer | | | Healthy | | | |
| Peak | 1 SS | 2 SS | 3 SS | 5 SS | 6 SS | 7 SS | 8 SS |
| 1 | 31 | 31 | 31 | 31 | 31 | 31 | 31 |
| 2 | 42 | 42 | 42 | 42 | 42 | 41 | 42 |
| 3 | 53 | 53 | 52 | 53 | 53 | 53 | 53 |
| 4 | 62 | 60 | 62 | 60 | 61 | 60 | 60 |
| 5 | 73 | 73 | 72 | 73 | 73 | 73 | 73 |
| 6 | 83 | 84 | 83 | 83 | 83 | 83 | 83 |
| 7 | 94 | 94 | 94 | 95 | 95 | 94 | 94 |
| 8 | 105 | 101 | 103 | 103 | 103 | 104 | 104 |
| 9 | 114 | 115 | 114 | 114 | 115 | 115 | 114 |
| 10 | 126 | 126 | 125 | 126 | 126 | 126 | 126 |
| 11 | 136 | 137 | 136 | 137 | 137 | 137 | 137 |
| 12 | 145 | 145 | 144 | 145 | 145 | 145 | 145 |
| 13 | 153 | — | 152 | — | — | — | — |
| 14 | 165 | 166 | 167 | 167 | 167 | 167 | 167 |
| 15 | 263 | 276 | 304 | 279 | 265 | 271 | 280 |

TABLE 2A
DSP sequencing

| | Threshold | |
|---|---|---|
| Size-based | for cancer | |
| screening parameter | screening | Screening |
| (bp) | positivity | capacity |
| Value at a specific | | |
| fragment size | | |
| 120 | >0.25 | ** |
| 130 | >0.32 | ** |
| 134 | >0.7 | *** |
| Difference | | |
| between values | | |
| 134-124 | >0.44 | *** |
| 155-145 | <0.25 | *** |
| Fragment size range | | |
| 40 to 145 | >20% | ** |
| 170 to 370 | <35% | * |
| 119 to 120 | >0.46 | *** |
| 145 to 250 | <80% | ** |
| 30 to 120 | >6% | ** |
| 145 to 250 | <80% | *** |
| 70 to 150 | >25% | ** |
| values ratio | | |
| 120 to 145/194 to 370 | >3 | ** |
| 119 to 120/194 to 370 | <0.06 | *** |
| 119 to 120/254 to 255 | >30 | *** |
| 30 to 120/145 to 250 | >0.08 | ** |
| 119 to 120/165 to 250 | >0.09 | ** |
| 100 to 145/145 to 250 | >0.21 | ** |
| Max peak/137 | <5 | ** |
| Max peak at 307/322 | >1 | ** |
| 156-145/156 | <0.25 | ** |
| Max peak/130 | <8.6 | *** |
| 307/322 | >0.8 | ** |

TABLE 2B
SSP vs DSP sequencing difference
as determined by delta S calculation

| Size-based | Threshold | |
|---|---|---|
| screening | for cancer | |
| parameter | screening | Screening |
| (bp/nt) | positivity | capacity |
| Difference between | | |
| values (%) | | |
| 110-80/H | >1 | ** |
| 100-90/H | >1 | ** |
| 155-125/H | >1 | **** |
| 90-155/H | <3 or | **** |
| | negative | |
| 130-160/H | >1 | **** |
| 120-150/H | >0.1 | *** |
| 120-150 | >0.5 | **** |
| size range values (%) | | |
| 70 to 150 cancer/H | >1 | ** |
| 70 to 145/H | >1 | ** |
| 40 to 145/H | >1.1 | *** |
| values ratio | | |
| 120 to 145/194 to 370 | >0.6 | * |
| 119 to 120/194 to 370 | >0.7 | *** |
| 119 to 120/254 to 255 | <0.7 | ** |

Table 2 A and B: Selection of specific size, difference of size or size ratio showing cancer screening capacity when comparing size profile obtained from single strand fragments (following cfDNA denaturation) or double strand fragments. Table 2A: selected value obtained with only DSP-S; and Table 2B, selected values obtained when directly comparing SSP-S and DSP-S. n bp to m bp=value of a fragment size range; n bp−m bp=difference of the value at two specific fragment size; n bp/m bp=ratio of the value at two specific fragment size; max peak=value at the peak showing the highest fragment number or %. /H=as compared to that of healthy reference value. Value can be expressed as sequencing reads, fragment number or % of the total fragment number. *, **, and ***, moderate, intermediate and high screening capacity. Indicative threshold for discriminating cancer and healthy subjects.

TABLE 3
Quantification of gain or loss of number
of fragments when assessing the difference
between cancer and healthy plasma and between
SSP-S vs DSP-S analysis. Gain and loss of
fragment number were calculated from the
curve of cumulative size frequency termed as
delta S. Peak means size corresponding to
the value of the maximal delta S.

| | | |
|---|---|---|
| Gain | Loss | Global gain |

D: Individual cancer minus
healthy mean from DSP-S

Sample 1

| | | | |
|---|---|---|---|
| Peak value | 152 | 220 | |
| S | 12.0 | −1.0 | 110 |

Sample 2

| | | | |
|---|---|---|---|
| Peak value | 164 | 230 | |
| Delta S | 8.9 | −0.9 | 8.0 |

Sample 3

| | | | |
|---|---|---|---|
| Peak value | 170 | 313 | |
| Delta S | 3.8 | 0.2 | 3.5 |

S: individual cancer minus
healthy mean from SSP-S

Sample 1

| | | | |
|---|---|---|---|
| Peak value | 144 | 230 | |
| Delta S | 21.8 | −0.1 | 21.7 |

Sample 2

| | | |
|---|---|---|
| Peak value | 162 | |
| Delta S | 13.3 | 13.3 |

Sample 3

| | | | |
|---|---|---|---|
| Peak value | 170 | 230 | |
| Delta S | 4.5 | −0.3 | 4.2 |

S-D: from SP-S minus DSP-S
(individual cancer and healthy mean)

Sample 1

| | | | |
|---|---|---|---|
| Peak value | 111 | 240 | |
| Defta S | 17.8 | −2.7 | 15.1 |

Sample 2

| | | | |
|---|---|---|---|
| Peak value | 120 | 250 | |
| Delta S | 11.1 | −1.3 | 9.8 |

Sample 3

| | | | |
|---|---|---|---|
| Peak value | 120 | 192 | |
| Delta S | 10.6 | −6.1 | 4.4 |

S-D: from SSP-S minus DSP-S
(individual cancer and healthy mean)

healthy mean

| | | | |
|---|---|---|---|
| Peak value | 152 | 202 | |
| Delta S | 9.2 | −4.9 | 4.3 |

TABLE 4
Frequency of various fragment size or fragment size range on seven samples
from healthy individuals and seven samples from CRC patients.

| Fragment | HEALTHY (n = 7) | | | | | | | | |
|---|---|---|---|---|---|---|---|---|---|
| size | % of fragments, DSP-S | | | | | | | | |
| values | Mean | SD | | | | | | | |
| (bp/nt) | healthy | healthy | 1 | 2 | 3 | 4 | 5 | 6 | 7 |
| 145 | 0.78% | 0.00086 | 0.83% | 0.77% | 0.65% | 0.84% | 0.83% | 0.69% | 0.88% |
| 166 | 2.42% | 0.00248 | 2.70% | 2.67% | 2.32% | 2.43% | 2.22% | 2.02% | 2.55% |
| 166/145 | 0.32 | 0.03391 | 0.31 | 0.29 | 0.28 | 0.34 | 0.37 | 0.34 | 0.35 |
| 30 to 220 | 87.28% | 0.02809 | 90.40% | 89.11% | 84.51% | 87.80% | 84.60% | 84.17% | 90.36% |
| 220 to 440 | 12.72% | 0.02797 | 9.61% | 10.90% | 15.50% | 12.17% | 15.35% | 15.82% | 9.66% |
| 30 to 145 | 13.40% | 0.01827 | 14.12% | 11.62% | 10.37% | 14.50% | 14.61% | 13.05% | 15.49% |
| 145 | 1.18% | 0.00095 | 1.19% | 1.29% | 1.12% | 1.25% | 1.16% | 1.00% | 1.22% |
| 166 | 1.86% | 0.00152 | 1.99% | 2.03% | 1.96% | 1.82% | 1.74% | 1.60% | 1.88% |
| 166/145 | 0.63 | 0.03845 | 0.60 | 0.63 | 0.57 | 0.69 | 0.67 | 0.63 | 0.65 |
| 30 to 220 | 95.16% | 0.01154 | 96.21% | 96.56% | 94.38% | 94.79% | 94.18% | 93.74% | 96.26% |
| 220 to 440 | 4.80% | 0.01136 | 3.77% | 3.43% | 5.59% | 5.16% | 5.75% | 6.21% | 3.72% |
| 30 to 145 | 33.08% | 0.02380 | 34.44% | 31.74% | 28.64% | 33.66% | 34.49% | 32.62% | 35.93% |

| Fragment | | | | | | | |
|---|---|---|---|---|---|---|---|
| size | | | | | | | |
| values | CANCER (n = 7) | | | | | | |
| (bp/nt) | 8 | 9 | 10 | 11 | 12 | 13 | 14 |
| 145 | 1.18% | 1.09% | 1.07% | 0.96% | 0.89% | 1.30% | 0.79% |
| 166 | 1.20% | 1.78% | 1.41% | 2.47% | 2.92% | 2.93% | 2.05% |
| 166/145 | 0.99 | 0.61 | 0.76 | 0.39 | 0.30 | 0.44 | 0.38 |
| 30 to 220 | 83.11% | 89.52% | 77.01% | 83.47% | 90.38% | 96.00% | 80.16% |
| 220 to 440 | 16.17% | 10.08% | 22.05% | 15.42% | 8.94% | 3.82% | 16.42% |
| 30 to 145 | 44.05% | 40.09% | 35.81% | 24.60% | 17.35% | 28.02% | 17.47% |
| 145 | 1.09% | 1.21% | 1.24% | 1.08% | 1.15% | 1.49% | 1.10% |
| 166 | 0.84% | 1.35% | 1.13% | 1.76% | 2.36% | 2.23% | 1.69% |
| 166/145 | 1.29 | 0.90 | 1.10 | 0.61 | 0.49 | 0.66 | 0.65 |
| 30 to 220 | 91.04% | 94.86% | 89.49% | 90.08% | 93.47% | 97.94% | 86.12% |
| 220 to 440 | 8.71% | 5.03% | 10.30% | 9.51% | 6.15% | 1.99% | 11.73% |
| 30 to 145 | 60.38% | 52.70% | 51.76% | 42.13% | 30.64% | 39.84% | 28.05% |

TABLE 5
Analyze size profile in particular from DSP-S in various cancers (breast,
liposarcoma and pancreas cancers) as compared to CRC. Here only two
discriminative factors, as revealed in the previous set (Table 2A)
are calculated to illustrate the capacity of size profile analysis to
provide discriminatory power between cancer and healthy individuals.

| cirDNA origin | 166/145 size ratio | 30 to 145% |
|---|---|---|
| Healty mean | 3.1 +/− 0.2 | 13.4 +/− 0.1 |
| Breast cancer | 2.2 | 14.6 |
| Pancreatic cancer | 1.4 | 16.0 |
| Liposarcoma cancer | 2.4 | 15.5 |
| Colorectal cancer | 1 to 1.5 | 17.4 to 44.1 |

TABLE 6
Determination of ΔV calculated from the percent frequency distribution data
as the difference between cancer patients and healthy individual mean from both DSP- or SSP-S
derived size profiles or as the difference between SSP-S vs DSP-S from both. Positive ΔV
values characterized cancer patient plasma.

| | ΔV for cancer minus healthy mean | | | | | | | | | | | |
|---|---|---|---|---|---|---|---|---|---|---|---|---|
| | DSP-S (bp) | | | | | | SSP-S (nt) | | | | | |
| | 8 | 9 | 10 | 11 | 12 | 14 | 8 | 9 | 10 | 11 | 12 | 14 |
| 40 to 80 | 5.76% | 4.86% | 2.46% | 1.98% | 0.96% | 1.28% | 5.89% | 0.94% | 0.16% | 2.63% | −1.07% | 4.25% |
| 40 to 120 | 18.17% | 17.11% | 10.85% | 6.43% | 3.01% | 3.40% | 19.45% | 11.79% | 8.26% | 5.66% | −2.80% | −4.27% |
| 40 to 160 | 29.96% | 27.69% | 21.41% | 14.15% | 6.40% | 3.32% | 22.05% | 18.06% | 16.05% | 8.46% | −0.79% | −6.13% |
| 80 to 120 | 12.74% | 12.57% | 8.54% | 4.56% | 2.13% | 2.20% | 13.84% | 10.94% | 8.12% | 3.15% | 4.82% | −3.11% |
| 130 to 160 | 8.10% | 7.54% | 6.85% | 6.64% | 3.32% | −0.21% | −0.26% | 3.48% | 4.18% | 2.15% | 2.37% | −1.40% |

TABLE 7
Comparison of size profile or ΔV values obtained when subtracting DSP-S values from SSP-S
values (SSP-S minus DSP-S) in 5 healthy and cancer individuals. Only negative ΔV are
obtained in the 40-160 range when subtracting healthy from cancer individual ΔV, as calculated here.
ΔV for SSP minus DSP

| Size range | 1 | 2 | 3 | 4 | 5 | 6 | 7 | 8 |
|---|---|---|---|---|---|---|---|---|
| 40 to 80 | 4.82% | 4.39% | 4.46% | 4.44% | 5.01% | 4.81% | 5.09% | 4.84% |
| 40 to 120 | 15.80% | 14.14% | 13.03% | 14.79% | 15.91% | 15.83% | 16.43% | 16.41% |
| 40 to 160 | 22.15% | 23.26% | 22.37% | 21.13% | 21.92% | 21.94% | 21.50% | 14.13% |
| 80 to 120 | 11.24% | 10.00% | 8.80% | 10.60% | 11.19% | 11.31% | 11.63% | 11.78% |
| 130 to 160 | 4.48% | 7.01% | 7.61% | 4.42% | 4.36% | 4.48% | 3.17% | −3.28% |
| Cancer minus | | | | | | | | −7.91% |
| healthy mean | | | | | | | | |
| (range 40 to 160) | | | | | | | | |

| Size range | 9 | 10 | 11 | 12 | 13 | 14 | Pvalue |
|---|---|---|---|---|---|---|---|
| 40 to 80 | 0.79% | 2.41% | 5.36% | 2.69% | 0.94% | 2.18% | 0.01294276 |
| 40 to 120 | 9.81% | 12.54% | 14.36% | 9.32% | 8.57% | 7.46% | 0.01209379 |
| 40 to 160 | 12.41% | 16.68% | 16.35% | 14.84% | 12.27% | 12.59% | 0.00000021 |
| 80 to 120 | 9.05% | 10.27% | 9.27% | 6.74% | 7.67% | 5.37% | 0.03910911 |
| 130 to 160 | 1.02% | 2.40% | 0.59% | 4.13% | 2.26% | 3.89% | 0.00903057 |
| Cancer minus | −9.63% | −5.36% | −5 69% | −7.20% | −9.77% | −9.45% | |
| healthy mean | | | | | | | |
| (range 40 to 160) | | | | | | | |

### EXAMPLES

Materials and Methods:

### Clinical Samples

Blood samples were collected from healthy individuals (IH, n=4) and colorectal cancer patient with stage IV (CRC, n=3). Samples from cancer patient were obtained from Cancer Institute of Montpellier (ICM, Val d'Aurelle, Montpellier, France). Samples from healthy individuals were obtained from Etablissement Francais du sang (EFS, Montpellier, France). All patients signed an informed consent and CRC samples from the ICM were obtained from the Nimes regional ethic committee. Samples were handled accordingly with a pre-analytical guideline previously established by our group.

Plasma Isolation and cfDNA Extraction

All samples were collected in 4-ml EDTA tubes. The blood was centrifuged at 1200 g at 4° C. for 10 min. The supernatants were isolated in sterile 1.5 ml Eppendorf tubes and centrifuged at 16 000 g at 4° C. for 10 min. Afterwards, the plasma was either immediately handled for DNA extraction or stored at −20° C. CfDNA was extracted from 1 ml of plasma using the QIAmp DNA Mini Blood kit (Qiagen) according to the “Blood and body fluid protocol”.(28) DNA extracts were kept at −20° C. until used.

Preparation of sequencing libraries Dual-indexed single-stranded libraries were prepared from 1-11 ng DNA from human plasma following the method described in (29) and using TL137 as a linker oligo. Dual-indexed double-stranded libraries were prepared from equal amounts of starting DNA as the single-stranded libraries. Double-stranded DNA End-repair was performed using the NEBNext End-Repair module (New England Biolabs, Ipswich, Mass.) in 39 ul total total volume and incubated 40 minutes at 20 C, followed by purification using Macherey-Nagel NGS Clean-up and Size Select beads (Macherey-Nagel GmbH & Co. KG, Duren, Germany) at 3× volume according to recommended protocol and eluted in 37 ul EBT (10 mM Tris pH 8 with 0.05% Tween 20). To allow sequencing with single-stranded libraries, custom double-stranded adapters were generated identical to those used in the single-stranded method by annealing a short oligo SLP4(29). Ligations were performed using NEBNext Quick Ligation Module (New England Biolabs) with 0.04 uM of each annealed adapter in 50 ul total volume and incubated for 30 minutes at 20 C. Adapters were elongated by adding 50 ul OneTaq 1× Master Mix (New England Biolabs) to the ligation mixture and incubating 20 minutes at 60 C. All oligos were acquired from Eurogentec (Kaneka Eurogentec, Seraing, Belgium). Both single- and double-stranded libraries were quantified via Q-PCR with a diluted fraction performed in a Lightcycler 2 (Roche Applied Science, Mannheim, Germany). Samples were then amplified using Taq polymerase an optimal cycles for each sample to avoid plateau phase, as calculated from the Ct of the diluted Q-PCR. Amplified samples were then purified by two rounds of Macherey-Nagel NGS beads at 1.3× volume in order to allow the retention of shorter inserts and eluted in 30 uL EBT. Purified libraries were then visualized on a Bioanalyzer 2100 (Agilent, Santa Clara, Calif.) and quantified via Q-PCR, Qubit 2 Fluorometer (Life Technologies, Grand Island, N.Y.), and Bioanalyzer 2100. Libraries were then pooled in equimolar amounts and sequenced on an Illumina MiSeq platform using two MiSeq v3 150 (2×75) kits, with sequencing primer CL72 (30) replacing the first read sequencing primer.

Size Profile Analysis by Deep Sequencing

All libraries were sequenced on MiSeq (Illumina, Reagent Kit v3 (150 cycle)). Adapter sequences were removed with cutadapt 1.3 (30) and reads were aligned to the human genome regference (hg19) with BWA aln (31) using the default parameters and filtered for mapping quality 20 with SAMtools 1.5 (32). Duplicate removal and histogram generation was performed with MarkDuplicates and CollectlnsertSizeMetrics, respectively, from Picard tools 1.88. We did a DNA size distribution profile of both libraries (DSP and SSP) from each sample. Cumulative frequency distribution analysis was done based on the data of size profile distribution. ΔS was calculated based on cumulative size frequency distribution data as the difference between cumulative of cancer patients and healthy individuals in DSP and SSP or as the difference between DSP VS SPP in both.

The chromosome normalization was adapted based on methods of Dreyfus (5). We calculated a normalization factor from each chromosome considering chromosome 1 as reference and we multiplied total reads by this factor independently for each chromosome to obtain relative reads to chromosome 1.

### Example 1: Use of Sequencing for Determining the Size Profile of cfDNA

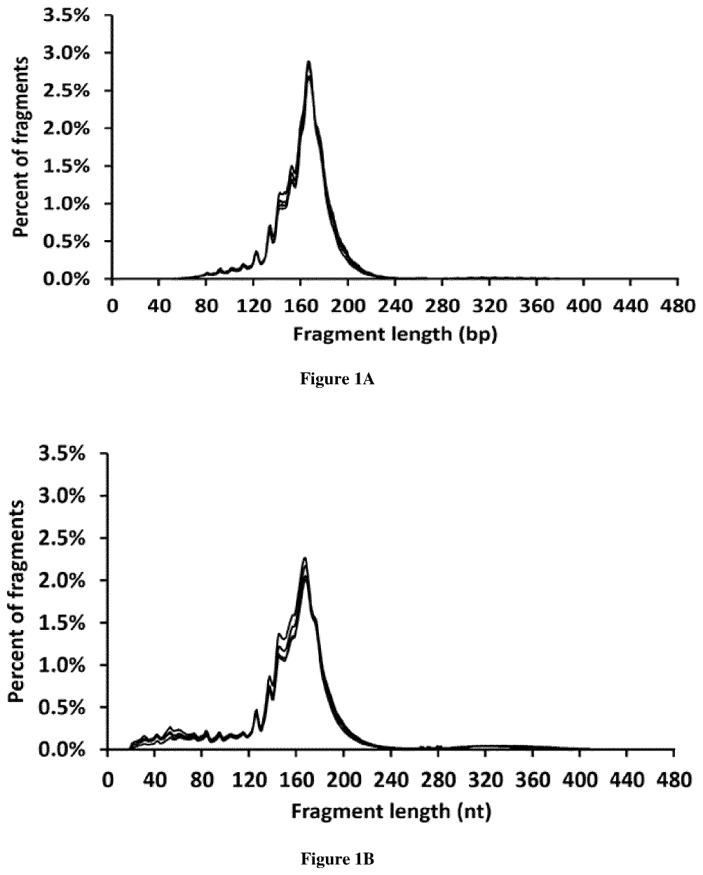

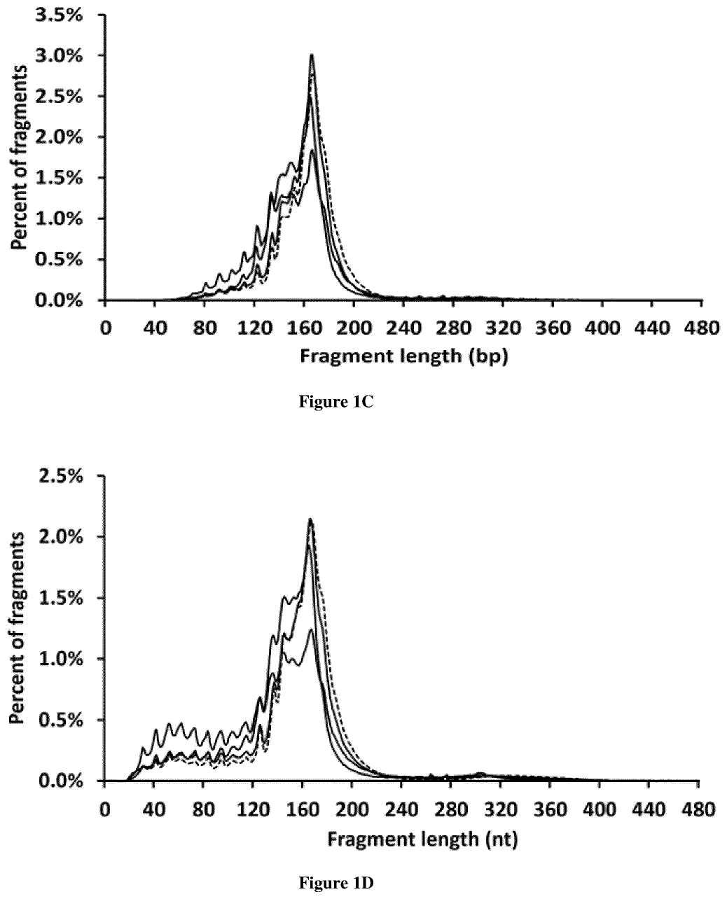

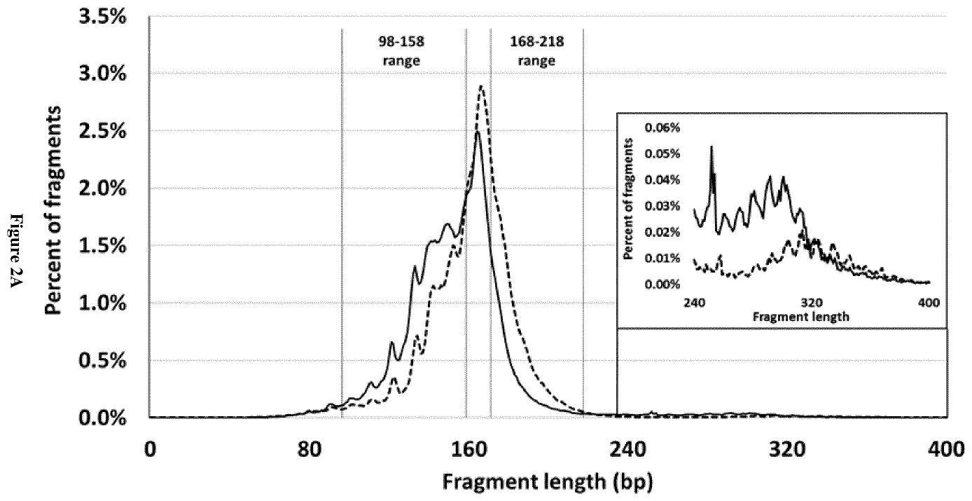

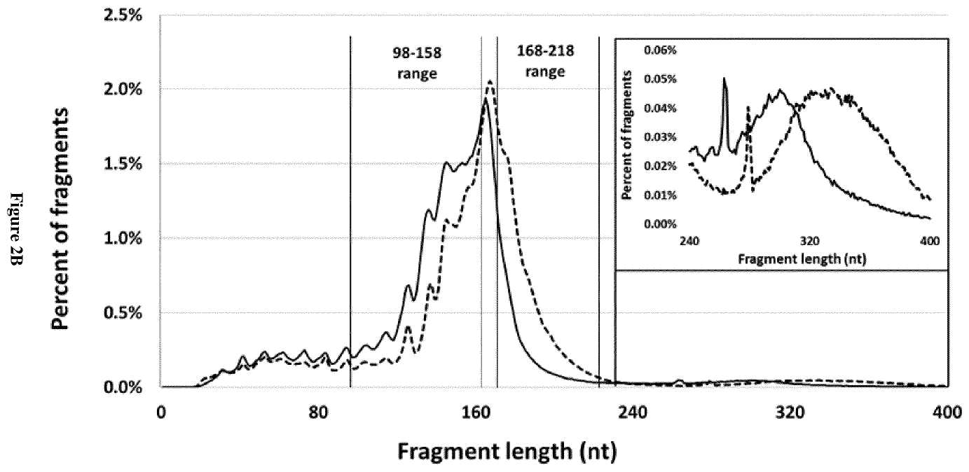

Sequencing can be carried out from a library preparation of either double-stranded DNA DSP) or single-stranded DNA (SSP). When applied to cfDNA both method show intimate variation of cfDNA size profile (10, 16). FIG. 1 shows size profiles of cfDNA from three cancer and four healthy human individuals obtained by both DSP- and SSP-based sequencing. Size profile of cfDNA showed very low variation as each subject curves superimposed when using both methods.

Healthy subject cfDNA size profile obtained by DSP sequencing (DSP-S) exhibited a mono-population (peaking at 166 bp and at 3% of fragments) ranging from 80 to 240 bp (FIG. 1A). Subpeaks of the four samples colocalized (Table 1). Healthy subject cfDNA size profile obtained by SSP sequencing (SSP-S) exhibited a population (peaking at 166 bp and at 2.2% of fragments) ranging from 120 to 240 bp; and a population plateauing at ˜0.2% from 25 to 120 bp (FIG. 1B). Subpeaks of the four samples colocalized (Table1). A very small population (peaking at 0.01%) is observable between 300 to 360 nt. When superimposing the mean size profile of the healthy individuals with that of each of the three cancer patients with using DSP-S, a subtle but reliable differences are observed (FIG. 1C): Cancer patient plasma showed higher amounts of fragments between 60 and 145 bp, but showed lower amounts between 166 and 240 bp, while both populations peaked at 166 bp. Note, a shoulder between 145 and 166 bp is observed in cancer patient plasma (FIG. 2A).

When superimposing the mean size profile of the healthy individuals with that of each of the three cancer patients with using SSP-S, overall the same subtle but reliable differences are observed (FIG. 1D): Cancer patient plasma showed higher amounts of fragments between 25 and 145 bp, but showed lower amounts between 166 and 240 bp, while both populations peaked at 166 bp. Note, the shoulder between 145 and 166 bp observed in cancer patients is slightly more pronounced in using SSP-S (FIG. 2B).

As previously observed in Thierry et al patent when using SSP-S, all those differences between healthy and cancer subjects increased with the mutant allele frequency (MAF) in sample 1, 2, and 3 (79.41%, 25.69%, and 7.15%, respectively) when using DSP-S. Note, the higher is MAF, the higher is the lower size fragments, and the lower is the level of the peak.

### Example 2: Use of Sequencing from Double Strand Library Preparation for Selecting Sizing-Based Screening Markers

In light of the previous patent from Thierry et al (34) based on the use of sequencing from single strand DNA library preparation of cfDNA, it is possible to use sequencing from double strand library preparation for selecting sizing-based marker to screen cancer patients among healthy individuals:

Selection of new calculation providing discrimination between cancer and healthy individuals are shown in Table 2A. Values observed at specific bp size (%), or obtained in calculating of specific size range (%), or obtained in calculating various values ratio are classified upon low (*), moderate (**) and high (***) screening capacity. A threshold is proposed for each selected sizing based screening marker.

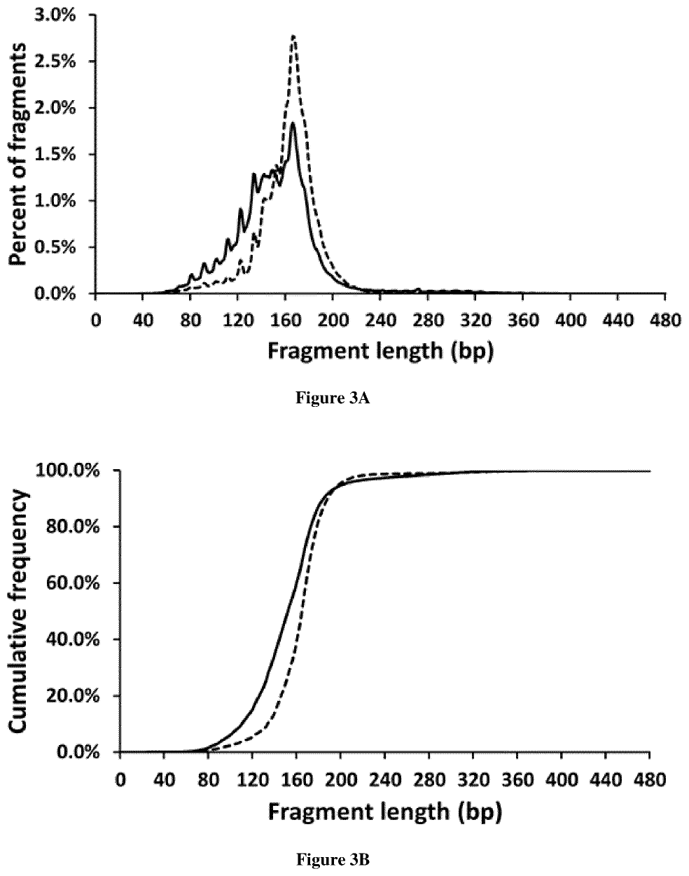

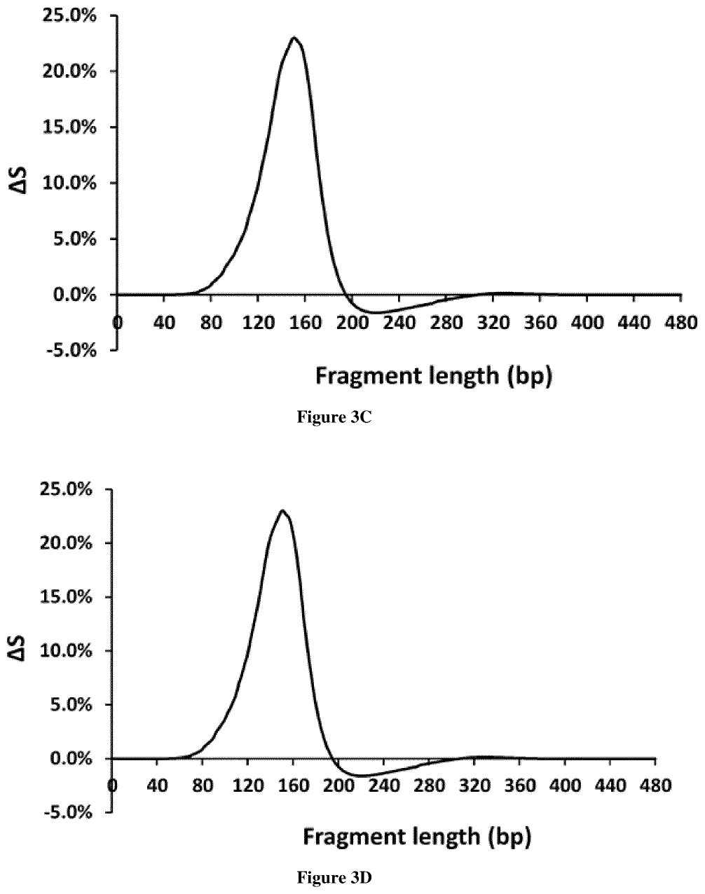

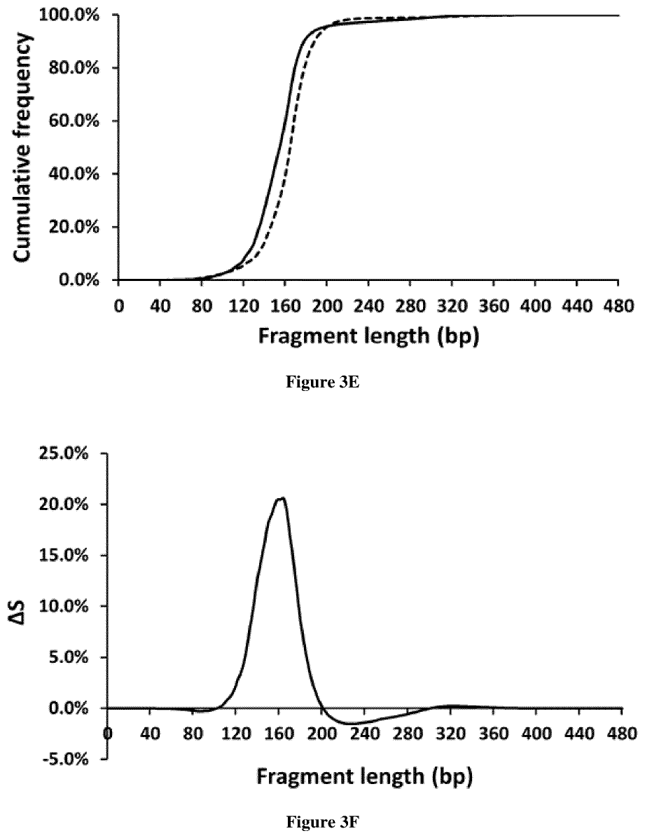

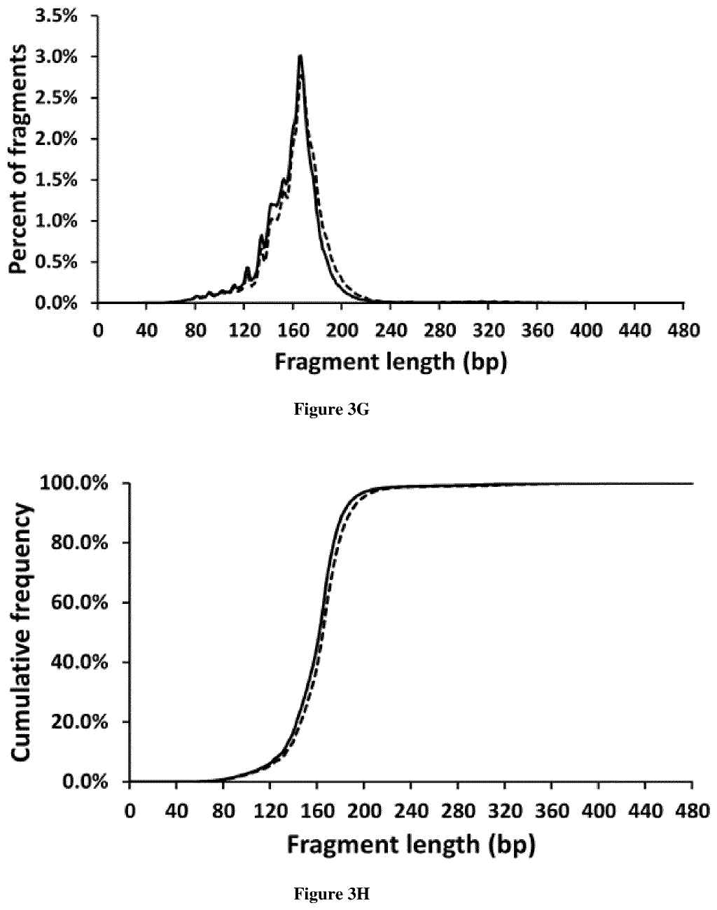

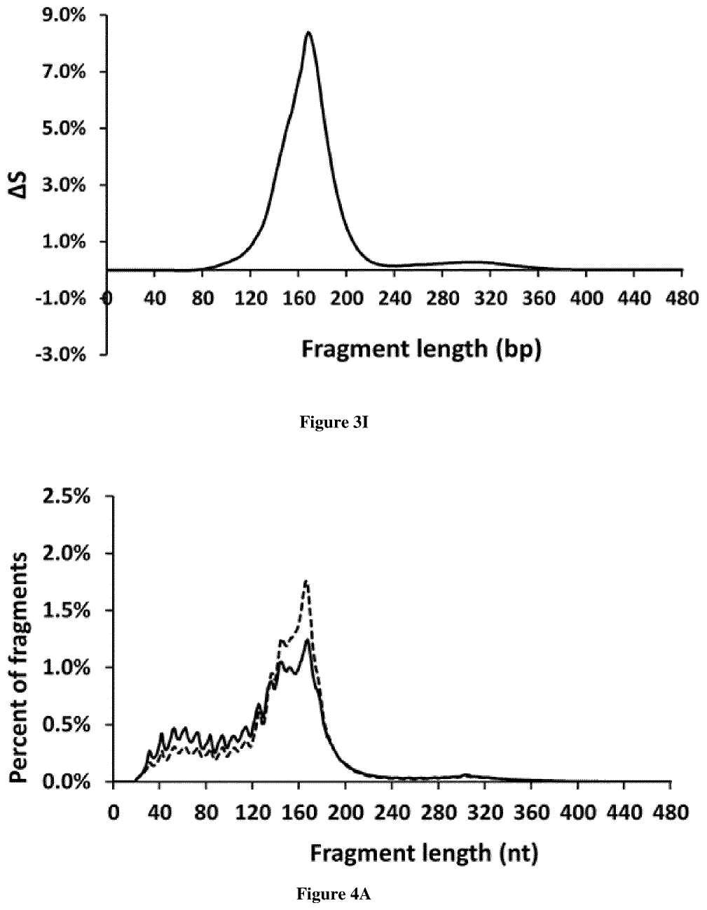

Selection was eased by determining from the size profile (FIG. 3A, D, G) cumulative values in function of fragment lengths (FIG. 3 B, E, H) and the difference between cancer patient and the healthy subject mean (calculated from #5, #6, #7 and #8 homogeneous values) cumulative values (FIG. 3 C, F, I). Two discriminating zones are revealed: a major difference between 80 and 200 bp peaking at 160 bp, and a minor difference between 200 and 320 bp peaking at 225 bp.

### Example 3: Previous Anteriority on the Use of Sequencing from Single Strand Library Preparation for Selecting Sizing-Based Screening Marker

Calculation providing discrimination between cancer and healthy individuals. Values observed at specific nt size (%), or obtained in calculating of specific size range (%), or obtained in calculating various values ratio are classified upon low (*), moderate (**) and high (***) screening capacity. A threshold is proposed for each selected sizing based screening marker.

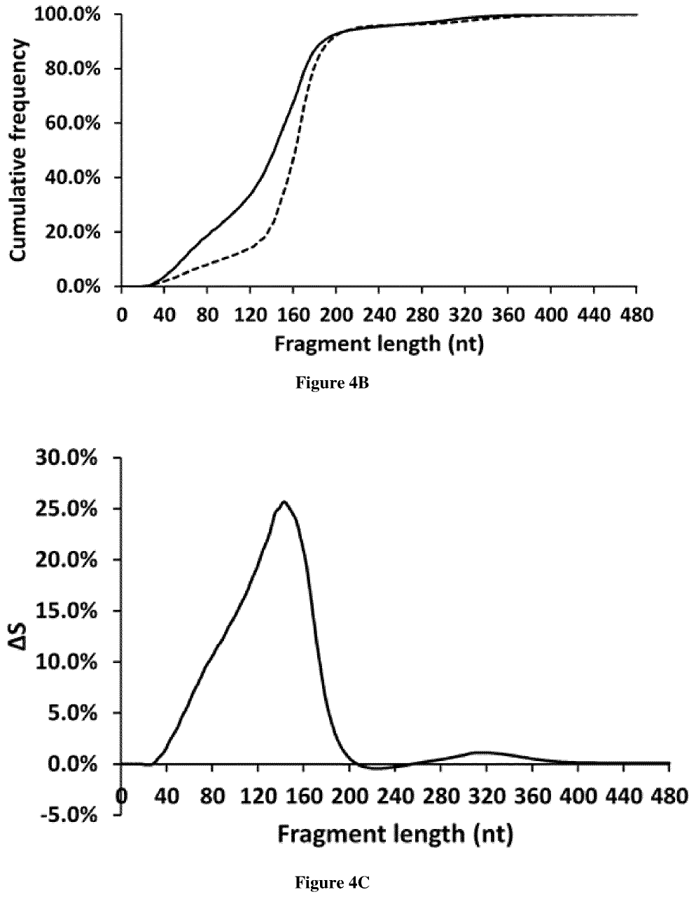

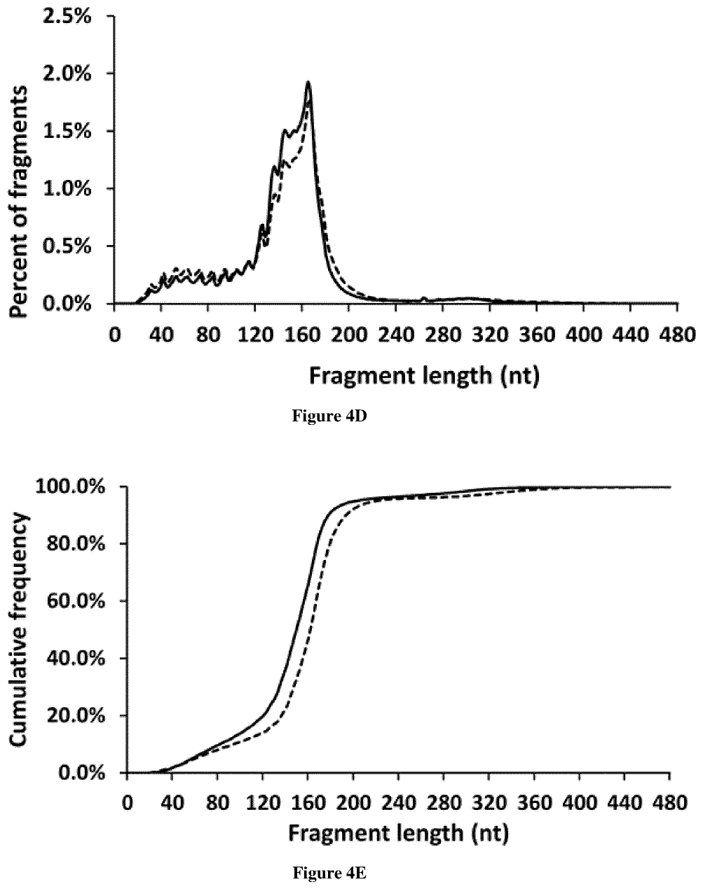

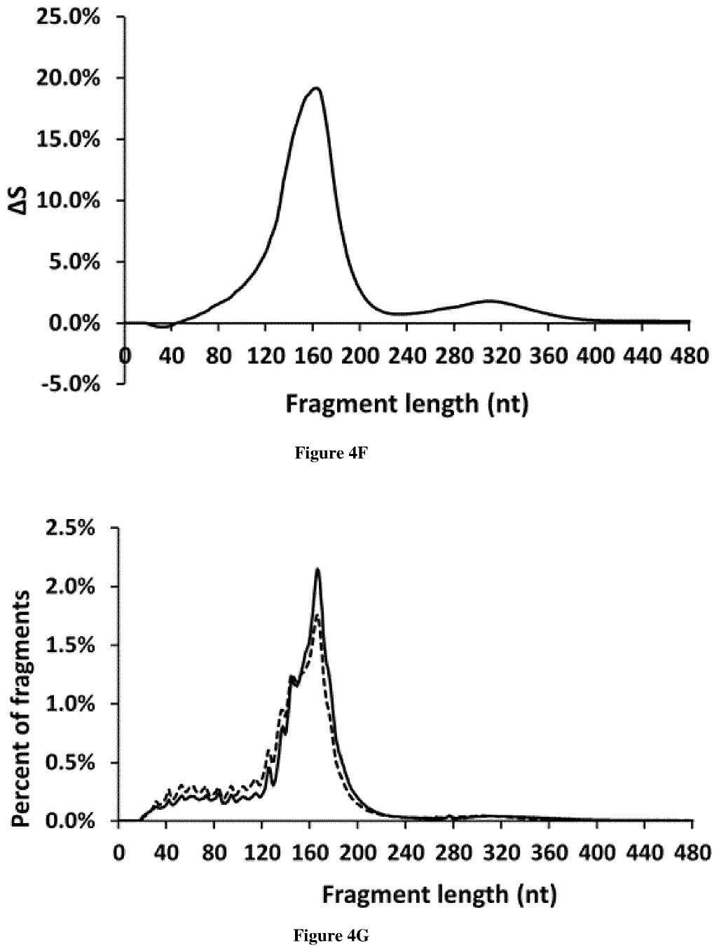

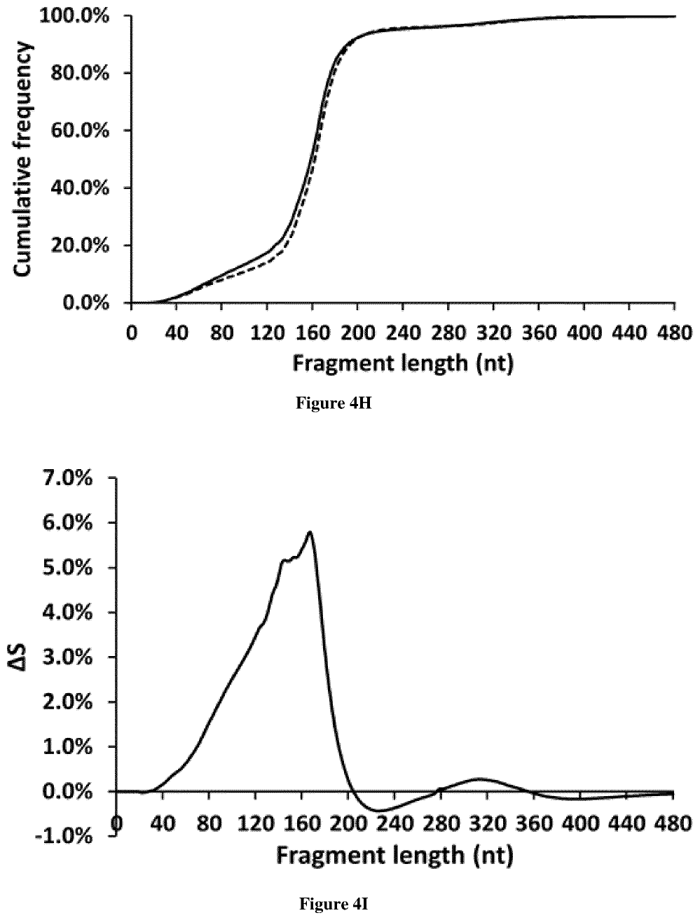

Selection was eased by determining from the size profile (FIG. 4A, D, G) cumulative values in function of fragment lengths (FIG. 4 B, E, H) and the difference between cancer patient and the healthy subject mean (calculated from #5, #6, #7 and #8 homogeneous values) cumulative values (FIG. 4 C, F, I). Two discriminating zones are revealed: a major difference between 40 and 200 nt peaking at 155 nt, and a minor difference between 220 and 390 nt peaking at 310 nt.

Note, there are clear differences in size profile obtained by DSP-S in as observed by the cumulative value differences between cancer patient and the healthy subject mean (FIG. 3). Table 2A sum up the most striking DSP-S differences with healthy when examining the percent of various fragment size range (in particular 80 to 145, 145 to 167, 167 to 200 and 307 to 322 ranges) or ration of, or difference at specific sizes.

### Example 4: Use of the Comparison Between the Size Profiles Obtained from SSP-S and DSP-S in Each Individual

Instead of only using either DSP-S or SSP-S, we can examining by both methods a plasma to screen and compare the value obtained from SSP to DSP. Since there are more fragments as determined by SSP-S the difference as expressed here as delta S from SSP-S over DSP-S.

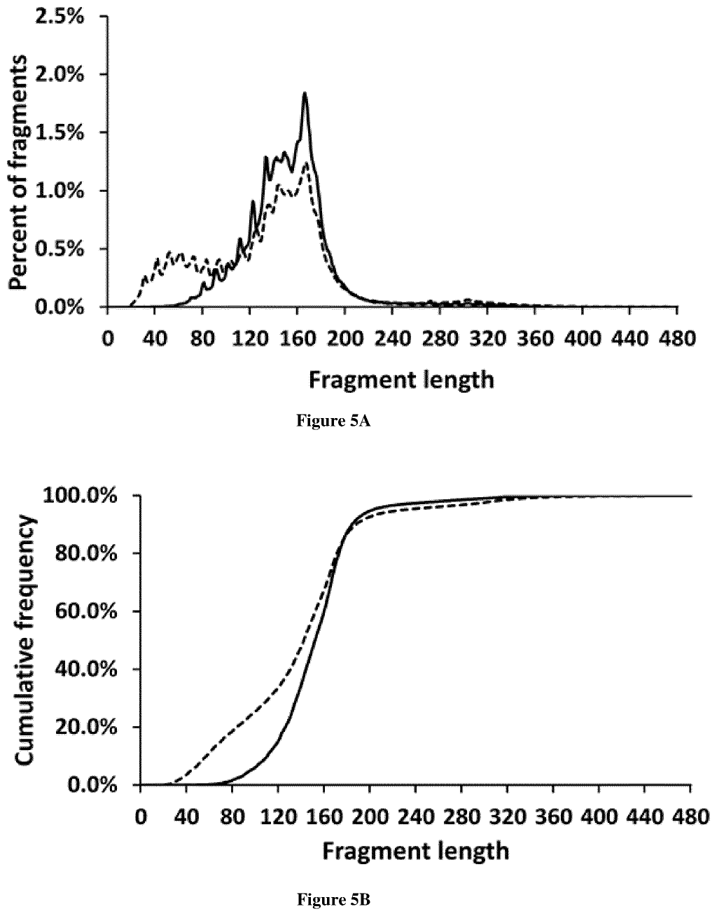

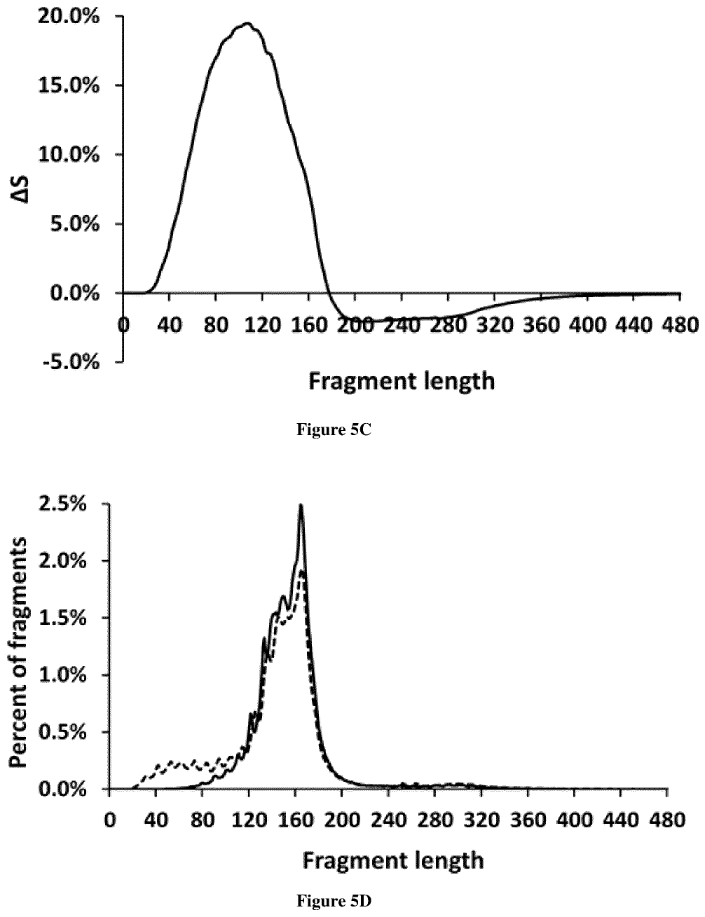

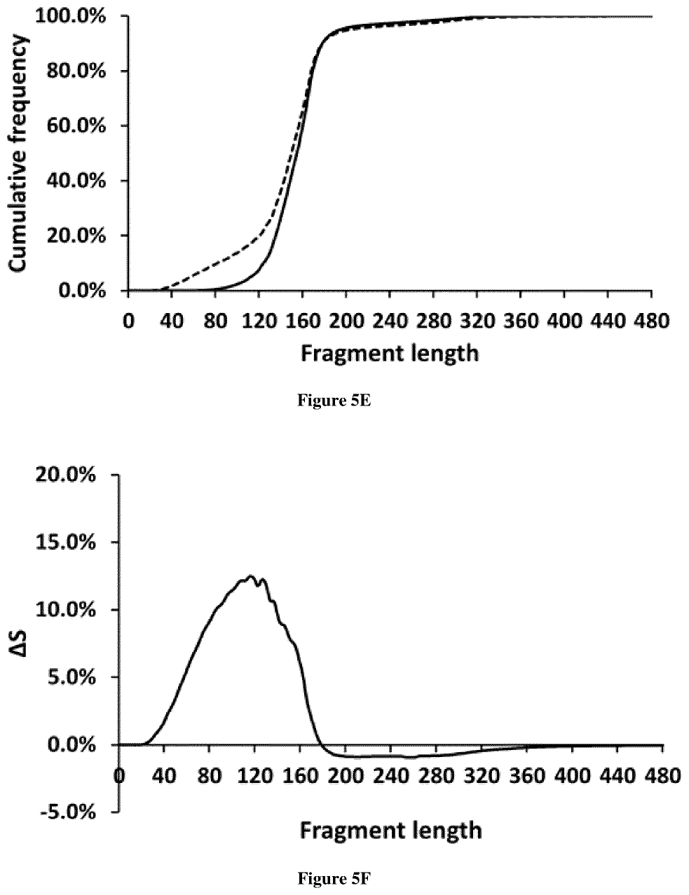

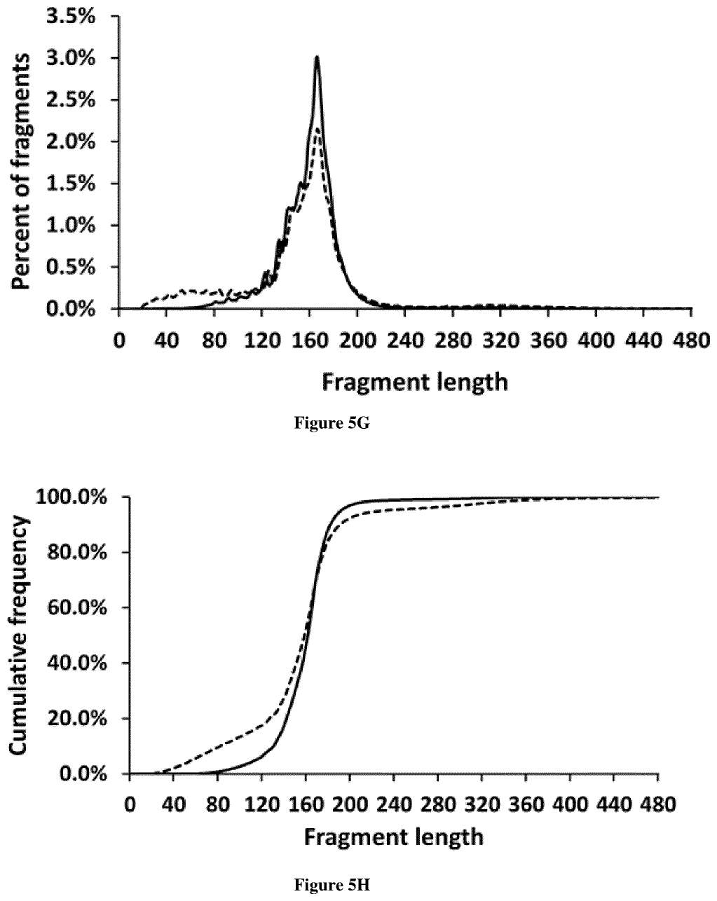

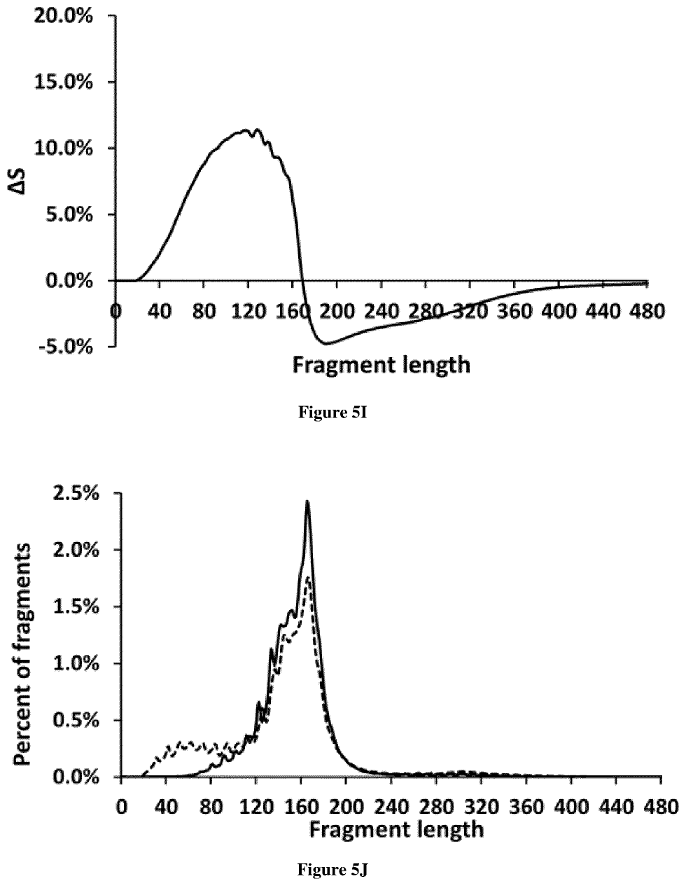

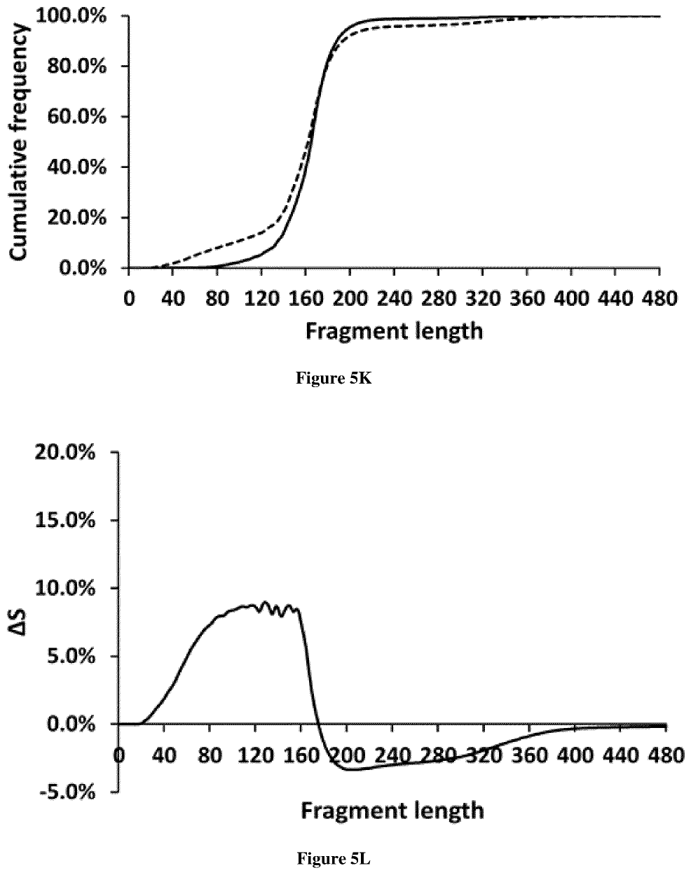

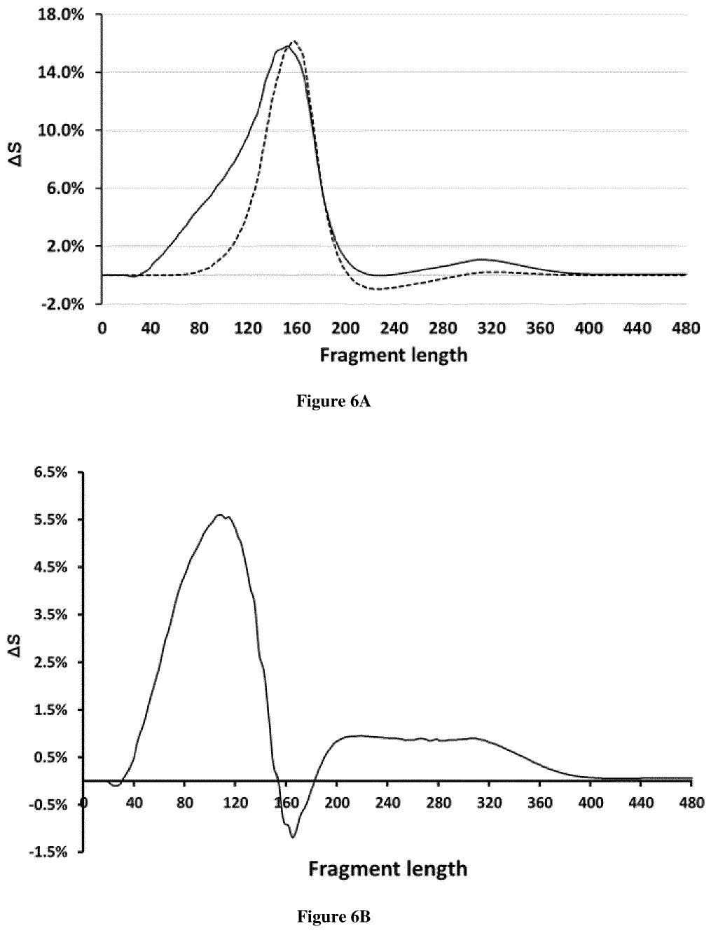

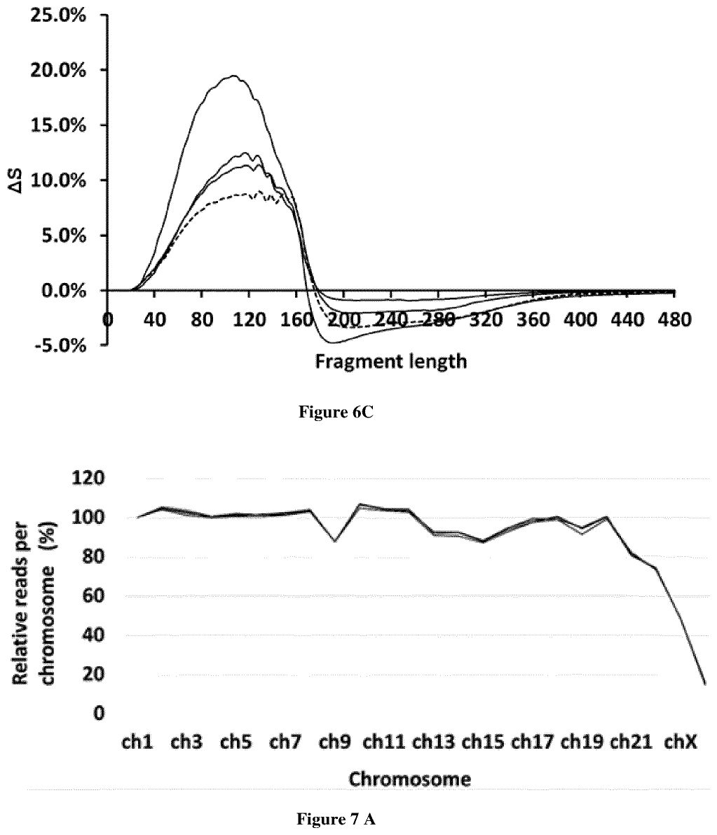

The difference of the size profile with that of healthy individual obtained for each cancer patient by SSP-S and DSP-S originating from determining the cumulative values are shown in FIGS. 3 and 4, respectively. FIG. 5 show the comparison of the difference (delta S or ΔS) between SSP (dotted line) and DSP (full line) analysis in each cancer patient (#1 FIG. 5A, B, C, #2 FIGS. 5 D, E, F and #3 FIG. 5 G, H, I) and in the mean values of the healthy subjects as above obtained (FIG. 5 J, K, L). Both cancer and Healthy individuals showed similar pattern revealing a positive difference (SSP-S minus DSP-S) for the 30 to 180 bp/nt size range, and a negative difference for the 180 to 440 bp/nt size range. Nonetheless, values obtained from healthy subjects (dotted curve) in FIG. 6C showed lower % at the peak (˜7.5%) when compared with #1, #2 and #3 (10, 11, and 19%). In addition, the curve for healthy plateaued from ˜110 to ˜150 nt in contrast to the curves obtained from all cancer patients. The later showed a maximum of 20%, 12% and 11%, in #1, 2, and 3, respective to their mutation frequency.

This highlights the possibility of using the difference between the size profile obtained from SSP-S and DSP-S when examining an individual to be screened. Table 2B sum up the selection of new calculation providing discrimination between cancer and healthy individuals. Values observed at specific bp/nt size (%), or obtained from the difference of the value at two fragment size or obtained in calculating of specific size range (%), or obtained in calculating various values ratio are classified upon low (*), moderate (**) and high (***) screening capacity. A threshold is proposed for each selected sizing based screening marker.

When carrying out both SSP-S and DSP-S on the same sample, the screening capacity may be very high (****, as revealed in Table 2B). Up to about 80 times discrimination between patient #1 and healthy mean when calculating the difference between value at 120 bp/nt and 150 bp/nt.

While a major and a very minor cfDNA peak are observable at ˜166 bp and ˜320 bp (Table 1), there are sub-peaks every ˜10 bp, due to intimate structure and association with histone octamers. Table 1, A-D, summarizes the detection of these sub-peaks in cancer and healthy individual cfDNA from either SSP or DSP library preparations. There are differences in sub-peaks, at specific cfDNA sizes, between sequencing techniques and between cancer and healthy individuals. Specific differences between the library preparations include subpeaks at 31, 42, 53, 60, 73 bp in healthy individuals that were only observed with SSP-S and not with DSP-S, whereas a subpeak at 152 bp was only seen with DSP-S. Note also that the SSP-S-derived subpeaks are ˜3 nt higher than those of DSP-S.

To sum up, DSP-S had subpeaks at 73 and 272 bp in cancer subjects and at 145 bp in healthy subjects that SSP-S did not. To summarize then, precise comparison of differences in subpeaks in both SSP- and DSP-S could potentially enable discrimination between cancer and healthy individuals.

### Example 5: Discrimination of Cancer Patient Plasma from Healthy Individuals by Analyzing the Size Profile Subpeaks

While a major and a very minor peak are observable at ˜166 bp and ˜320 bp (table 1), cfDNA size profile harbor every ˜10 bp subpeaks due to the intimate structure and association with histone octamers. Table 1 summarize the detection of these subpeaks in either cancer and healthy individual cfDNA size profile obtained from either SSP or DSP library preparations. They are a few differences in the presence of subpeaks at specific cfDNA size between either library preparation types or between cancer and healthy individuals:

Differences in the presence of subpeaks at specific cfDNA size between SSP and DSP library preparations:
In healthy individuals we observe subpeaks at 31, 42, 53, 60, 73 following only SSP-S and not DSP-S.
The subpeak at 152 bp exist only following DSP-S.
Note, SSP-S derived subpeak fragment size are ˜3 bp higher than that of DSP-S

Differences in the presence of subpeaks at specific cfDNA size between cancer and healthy subjects upon library preparations:
In contrast to SSP-S, DSP-S showed subpeaks at 73 and 272 bp in cancer subjects
In contrast to SSP-S, DSP-S showed a subpeak at 145 bp in healthy subjects

Consequently, precise and comparative observation of subpeak presence when using both SSP- and DSP-S enable to discriminate cancer to healthy individuals.

### Example 6: Selection of Specific Size, Difference of Size or Size Ratio Showing Cancer Screening Capacity when Comparing Size Profile Obtained from Single Strand Fragments (Following cfDNA Denaturation) or Double Strand Fragments

Table 2A shows selected value obtained with only DSP-S; and Table 2B, selected values obtained when directly comparing SSP-S and DSP-S. They are values with moderate, intermediate or high screening capacity. Indicative threshold for discriminating cancer and healthy subjects are proposed.

The invention propose to combined those selective values (especially the ones with high screening capacity), to improve reliability of the test. It is possible to elaborate an algorithm towards this goal.

### Example 7: Comparing Healthy to Cancer Plasma by Determining the Fragmentation Level Per Chromosome

Method: Determination of the Fragmentation Level Per Chromosome by Mappability Examination

The number of reads, per sample, mapping to each chromosome, irrespective of length of the reads were determined from analyzing sequencing mappability. As the number of reads per chromosome correlates with the number of fragments, it consequently correlates to the level of fragmentation per chromosome (higher fragmentation infers higher number of shorter fragments). These values are the expected/observed number of reads mapping to each chromosome for a diploid genome. We performed this analysis as a chromosome-level ploidy check in which a value of unity means that particular chromosome is neither over- or underrepresented, for a normal diploid genome. A value of 0.5 for the X indicates of a single X chromosome, thus a male. Note, this analysis does not take into account any differences in the proportion of the reference genome that is mappable; differences in mappability introduce a small variation between chromosomes away from all being exactly at a value of 1. The chromosome normalization was adapted based on methods of Dreyfus (30). We calculated a normalization factor from each chromosome considering chromosome 1 as reference and we multiplied total reads by this factor independently for each chromosome to obtain relative reads to chromosome 1. For clarity all values in the figures are plotted relative to that of chromosome 1 (100%).

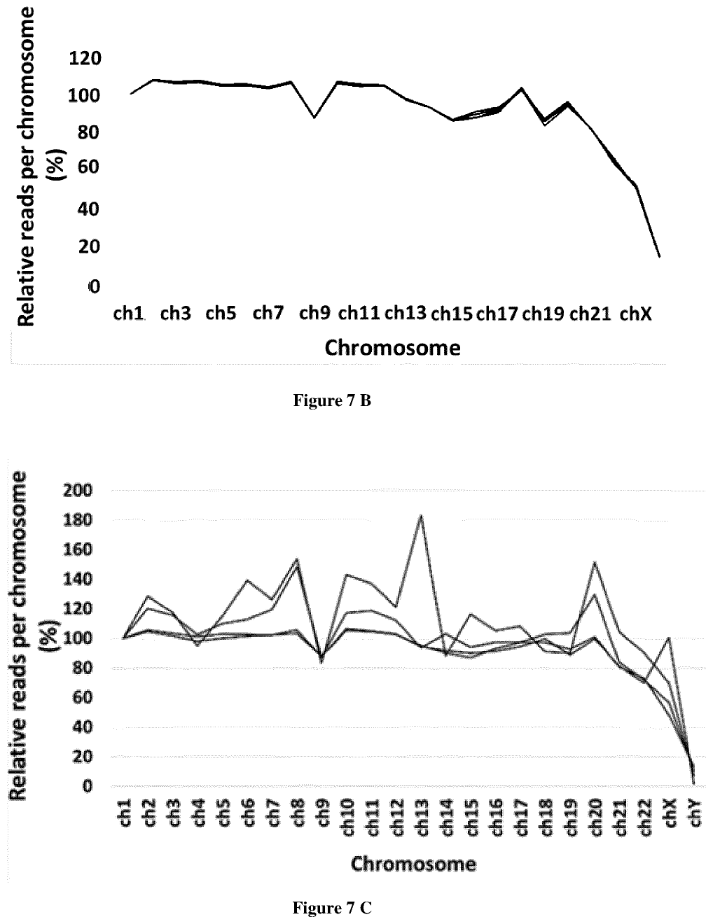

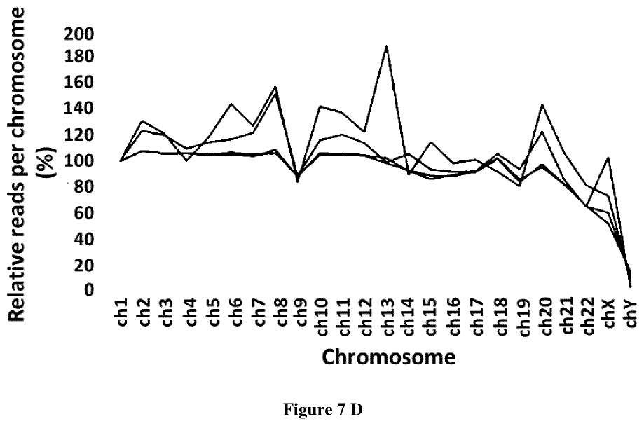

Results: FIG. 7 indicates the relative values of the cfDNA reads per chromosome we obtained with DSP-S. CfDNA mappability varied little between all the autosomal chromosomes, and there was the anticipated decrease in values for chX and chY respective to the gender of the individuals which are all male. Note, the relative reads per chromosomes were equivalent with using either DSP-S(A) or SSP-S(B).

When comparing the chromosome mappability, only the cfDNA from the mCRC patients with the two highest MAF had clear and common variations compared to the healthy individuals. Higher reads were found for ch2, 6, 8, 10, 11, and 20. In particular, more than a 20% increase of fragmentation was observed in ch2, 8 and 20. As for healthy subject derived cfDNA, the relative reads per chromosomes were equivalent with DSP-S and SSP-S (PCT 2017). Note, there was the anticipated decrease in values for chX and chY respective to the gender of sample #1, 2, and 3 (male) and no variation (˜100%) for chX for the #4 female patient.

Conclusion: Our data suggest that the determination of the fragmentation level per chromosome by mappability examination could be a potential biomarker of the cfDNA deriving from malignant cells. In addition, our data demonstrated as reported earlier that fragmentation is globally rather uniform along the genome in healthy derived cfDNA, and alternatively heterogenous upon DNA region (WO/2019/110750, Cristiano). Our study first described that higher fragmentation occurs in specific chromosomes such as chr2, 8 and 20. This may be explain in the context of the overall higher fragmentation of cfDNA deriving from malignant cells as observed here and elsewhere. We can speculate that there are intrinsic genomic factors exposing the DNA molecule to or leading to higher nuclease sensitivity (i.e. histone acylation, transcription factor binding site, . . . ), or extrinsic genomic factors resulting from the specific biological environment of the malignant cells (i.e. increased and/or different nuclease activity (10), higher viscinity with lymphocytic cells, . . . ), or the specific and high proportion in the tumor of highly proliferative cells resulting to higher accessibility of the chromatin. Counting fragment per chromosome (for determining the fragmentation level per chromosome) in comparison with reference chromosome appear as a powerful tool to characterize malignant cell derived cfDNA and consequently potentially enable cancer screening.

### Example 8: Comparing Healthy and Cancer Subject Plasma by Plotting the Cumulative Size Frequency of a Cancer Patient Plasma with a Healthy Derived Plasma

Method: Cumulative size frequency distribution analysis was performed based on the size profile distribution data. ΔS was calculated from the cumulative size frequency distribution data as the difference between cancer patients and healthy individuals (Example 1).

Results: The difference of cumulative size frequency (ΔS value) is smallest for the mCRC cases with the lowest MAF, using DSP-S(9, 21 and 24%). When further examining ΔS values, we observed gain and loss of fragments resulting in a global gain of 11.0%, 8.0, and 3.5%). This revealed that global gain increases with MAF. In addition the fragment size at the maximal gain appeared indirectly proportional to the MAF. ΔS gain greatly increased with the % of malignant cell derived cfDNA (up to 12%); 1% being considered as a threshold for screening cancer; and (2), A S global gain greatly increased with the % of malignant cell derived cfDNA (up to 11%); 1% being considered as a threshold for screening cancer. ΔS loss is for all cancer plasma inferior to 1%.

Conclusion: Plotting the cumulative size frequency (ΔS values) with using reference values for healthy, enables to discriminate cancer vs healthy plasma by various ways by comparing to healthy individuals: (i), the size at the maximal ΔS value; (ii), the maximal ΔS value; and (iii), the ΔS gain and the ΔS loss.

### Example 9: Plotting the Cumulative Size Frequency (ΔS Values) as Obtained by SSP-S and DSP-S from the Difference of a Cancer Patient Plasma with a Healthy Derived Plasma in the Same Plasma

Method: Comparing cumulative size frequency as obtained by SSP-S and DSP-S from the difference of a cancer patient plasma with a healthy derived plasma in the same plasma Results: We directly compared the performance of the two sequencing techniques by calculating the differences in the cumulating frequencies, denoted as ΔS (Example 4). There was for healthy individuals a plateau between 80 and 160 bp corresponding to 9% ΔS (FIG. 5). The curve of A S plotted vs fragment lengths showed a gain and a loss of amount of fragments of 9.2% and 4.9% peaking at 152 bp and 202 bp, respectively, revealing an overall gain of 4.3% (Table 3).

Next, we directly compared the performance of the two sequencing techniques for each of the three cancer patients (Example 4). Overall, cfDNA fragments from 30 to 80 nt are nearly only detectable by SSP-S(91.2% as compared to DSP-S examination). Second, there is a slight difference between SSP-S and DSP-S for fragments in the range between 120 to 240: For the healthy individuals there were 5.6% more cfDNA fragments between 80 and 166 nt and 19.6% less between 166 to 240 nt when examining SSP-S as compared to DSP-S (FIG. 5). The greatest differences were seen in the high-MAF mCRC patients: We observed an increase of fragment number within the 80 and 166 nt range of 12.2, 8.6 and 22.3% and a slight decrease of 17.9, 20.7 and 30.4% within 166 to 240 nt range in #4, 3, 2, 1 mCRC patient plasma. ΔS was maximal, for both cancer and healthy subjects, at ˜120 bp. Note, the ΔS curve is sharper for subjects with higher MAF where significant differences could be observed (20%, 12%, 11% for #1, 2 and 3). In contrast, there was a plateau between 80 and 160 bp in healthy subject cfDNA corresponding to 9% ΔS (FIG. 4). ΔS peaking at ˜120 bp appeared as a landmark of cfDNA cancer patients as compared to healthy individuals (˜150 bp). When further examining ΔS values, we observed gain and loss of fragment number resulting in a global gain of 15.1%, 9.8%, and 4.4% in sample #1, 2 and 3, respectively. This revealed that global gain, as observed when comparing SSP-S with DSP-S, increases with MAF.

Conclusion: Plotting the cumulative size frequency (ΔS values) as obtained by SSP-S and DSP-S from the difference of a cancer patient plasma with a healthy derived plasma in the same plasma enabled to discriminate cancer vs healthy plasma by various ways by calculating the difference of DSP-S ΔS values from SSP-S ΔS values: (i), the size at the maximal ΔS value; (ii), the maximal ΔS value; and (iii), the ΔS gain and the ΔS loss.

Conclusion:

Using conventional whole-genome sequencing of a double-stranded as shown with single-stranded DNA library and Q-PCR (former PCT) offers an opportunity to definitively set cfDNA size profile and decipher structural feature of the cell free DNA circulating in human blood. Therefore, the inventors compared the structure of cfDNA in healthy individuals and cancer patients. This exploratory study was based on the blinded examination of four plasma from healthy individuals and the plasma of four metastatic colorectal cancer (mCRC) patients presenting a wide variation of mutant allele frequency (7.1%, 25.7% and 79.4%). They compared the cfDNA size distribution in these healthy and mCRC subject plasma as determined by using whole genome sequencing. This study showed that detection of cfDNA from cancer cells is possible by examining cfDNA size profile and calculating the respective proportion of the number of fragments specific size range(s) in a single cancer plasma and the corresponding mean value of healthy plasma.

Considering the anteriority, the innovation of this patent description relies on:
Determination of precise calculation and combination thereof (comparison with the number of fragments from healthy references at specific sizes, size ranges, size differences, size ratio, size range ratio) from DSP-S.
Comparison of a tested cfDNA size profile with that of reference healthy subject with using the cumulative size frequency analysis from DSP-S.
Directly comparing the analysis by DSP-S with SSP-S in a single plasma and determining precise calculations and combination thereof (comparison with the number of fragments from healthy references at specific sizes, size ranges, size differences, size ratio, size range ratio) as well as cumulative frequency analysis.
Determining the number of fragments per chromosomes and detecting whether chromosome 2, 6, 8, 10, 11 and 20 show higher fragmentation as discriminating criteria to screen cancer.

### Example 10: Analysis on Seven Samples from Healthy Individuals and Seven Samples from CRC Patients

We carried out a second set of analysis on seven samples from healthy individuals and seven samples from CRC patients. The results are equivalent to the previous set of samples. Table 4 described the frequency of various fragment size or fragment size range found in these samples. The 166/145 fragment size frequency ratio derived from DSP-S showed discriminative power between healthy (0.32+/−0.03 SD) and the seven CRC samples (0.38 to 0.99). When using SSP-S analysis the 166/145 fragment size frequency ratio is 0.63+/−0.38 and ranging from 0.49 to 1.29 in mean healthy and the seven CRC samples, respectively (data not shown). Moreover, the fragment size frequency of the 30-145 bp range as compared to the total fragment size within 30-440 bp (corresponding to DNA in mono- and di-nucleosomes) calculated from DSP-S showed discriminative power between healthy (13.40+/−0.02 SD) and the seven CRC samples (17.4 to 44.05). When using SSP-S analysis, the fragment size frequency of the 30-145 bp range is 33.08+/−0.02 and ranging from 28.05 to 60.38 in mean healthy and the seven CRC samples, respectively (data not shown).

### Example 11

We found similar observations and discrimination by analyzing size profile in particular from DSP-S in various cancers (breast, liposarcoma and pancreas cancers) as compared to CRC. The discriminating parameters as previously highlighted enable to distinguish these cirDNA from healthy individuals. Table 5 shows data on two of the parameters with the strongest discriminatory power for each cirDNA extract cancer. Values from calculating the 166/145 bp ratio and the 30-145 size range % obtained from cancer plasma are significantly different as compared with those obtained from the mean of seven healthy individuals plasma. All the values found for 166/145 ratio from cancer individuals are lower than that of healthy mean individuals. All the values found for 30-145 bp from cancer individuals are higher than that of healthy mean individuals. The very high reproducibility of the size profile when examining healthy individual plasma (i.e. 166/145 ratio and 30-145 ratio, 3.1+/−0.2 and 13.4+/−0.1) enables to consider the observed difference as significant. These data showed that deeply analyzing size profile and calculating the parameters as described here are valuable to all cancers in screening for cancer.

### Example 12

We termed ΔV the calculation from the percent frequency distribution data as the difference between cancer patients and healthy individuals in either DSP and SSP or as the difference between SSP vs DSP in both. AV appears as a strong discriminatory parameter as observed in Table 6. When calculated from size ranges, 40-160 bp showed the highest differences in plasma from six colorectal cancer of various MAF (0.9, 14.3, 23.3, 47.3, 54.6 and 68.5%, numbered 14, 12, 11, 10, 9 and 8) with that of healthy mean from seven healthy individuals (Table 6). The percentage or difference increased with MAF, demonstrating the increased presence of malignant cell derived cirDNA and consequently confirm the validity of this size profile parameter. Thus, positive ΔV values characterized cancer patient plasma DNA in various size ranges between 40 and 160 bp (nt) (Table 6). AV in the 40-160 size range showed the highest ΔV values and varied from 3.32% to 29.96% and from −6.13 to 22.05% (when using DSP-S and SSP-S, respectively). Note, ΔV is always negative between 160 and 220 bp (nt) but difference is minor as compared to 40-160 bp (nt) range, then lesser discriminative (data not shown). AV appeared as a discriminatory factor when comparing cancer vs healthy individuals.

### Example 13

We already above described that a significant difference exist between cirDNA from healthy and cancer individuals when comparing size profile or ΔS values obtained from SSP-S and DSP-S(SSP-S minus DSP-S). The same observation is made when comparing size profile or ΔV values. For instance, ΔV calculated within the 40 to 160 bp/nt range from the mean of seven healthy plasma is 22.04+/−0.68 SD (difference in frequency, %); and ΔV of the seven plasma from CRC patients varied from 12.27 to 16.68% (Table 7). The P value (<0.0001) showed that a strong statistical difference exist between both groups of individuals when comparing SSP-S and DSP-S size profile values. Only negative ΔV values are obtained in the 40-160 range when substracting ΔV healthy values from cancer individual ΔV values. ΔV<20% revealed values obtained from plasma cancer subjects. ΔV calculated when substracting DSP-S % from SSP-S % are discriminative when comparing cancer to healthy plasma DNA.

### REFERENCES

Throughout this application, various references describe the state of the art to which this invention pertains. The disclosures of these references are hereby incorporated by reference into the present disclosure.
1. Mouliere, F., Robert, B., Peyrotte, E. A., Rio, M. D., Ychou, M., Molina, F Gongora, C. and Thierry, A. R. (2011) High Fragmentation Characterizes Tumour-Derived Circulating DNA. PLOS ONE, 6, e23418.
2. Straver, R., Oudejans, C. B. M., Sistermans, E. A. and Reinders, M. J. T. Calculating the fetal fraction for noninvasive prenatal testing based on genome-wide nucleosome profiles. Prenatal Diagnosis, 36, 614-621.
3. Thierry, A. R., El Messaoudi, S., Gahan, P. B., Anker, P. and Stroun, M. (2016) Origins, structures, and functions of circulating DNA in oncology. Cancer Metastasis Rev., 35, 347-376.
4. Stroun, M., Anker, P., Maurice, P., Lyautey, J., Lederrey, C. and Beljanski, M. (1989) Neoplastic characteristics of the DNA found in the plasma of cancer patients. Oncology, 46, 318-322.
5. Wan, J. C. M., Massie, C., Garcia-Corbacho, J., Mouliere, F., Brenton, J. D., Caldas, C., Pacey, S., Baird, R. and Rosenfeld, N. (2017) Liquid biopsies come of age: towards implementation of circulating tumour DNA. Nat Rev Cancer, 17, 223-238.
6. Diaz, L. A. and Bardelli, A. (2014) Liquid biopsies: genotyping circulating tumor DNA. J. Clin. Oncol., 32, 579-586.
7. Mouliere, F., Messaoudi, S. E., Gongora, C., Guedj, A.-S., Robert, B., Rio, M. D., Molina, F., Lamy, P.-J., Lopez-Crapez, E., Mathonnet, M., et al. (2013) Circulating Cell-Free DNA from Colorectal Cancer Patients May Reveal High KRAS or BRAF Mutation Load. Translational Oncology, 6, 319.
8. Bianchi, D. W. and Chiu, R. W. K. (2018) Sequencing of Circulating Cell-free DNA during Pregnancy. New England Journal of Medicine.
9. Lo, Y. M., Tein, M. S., Lau, T. K., Haines, C. J., Leung, T. N., Poon, P. M., Wainscoat, J. S., Johnson, P. J., Chang, A. M. and Hjelm, N. M. (1998) Quantitative analysis of fetal DNA in maternal plasma and serum: implications for noninvasive prenatal diagnosis. Am J Hum Genet, 62, 768-775.
10. Sanchez, C., Snyder, M. W., Tanos, R., Shendure, J. and Thierry, A. R. (2018) New insights into structural features and optimal detection of circulating tumor DNA determined by single-strand DNA analysis. npj Genomic Medicine, 3, 31.
11. Underhill, H. R., Kitzman, J. O., Hellwig, S., Welker, N. C., Daza, R., Baker, D. N., Gligorich, K. M., Rostomily, R. C., Bronner, M. P. and Shendure, J. (2016) Fragment Length of Circulating Tumor DNA. PLoS Genetics, 12.
12. Methods in Enzymology (1980) In Methods in Enzymology, Nucleic Acids Part I. Academic Press, Vol. 65, pp. xi-xvi.
13. Maxam, A. M. and Gilbert, W. (1977) A new method for sequencing DNA. PNAS, 74, 560-564.
14. Tabor, S. and Richardson, C. C. (1987) DNA sequence analysis with a modified bacteriophage T7 DNA polymerase. PNAS, 84, 4767-4771.
15. Bennett, E. A., Massilani, D., Lizzo, G., Daligault, J., Geigl, E. M. and Grange, T. (2014) Library construction for ancient genomics: single strand or double strand? Biotechniques, 56, 289-300.
16. Burnham, P., Kim, M. S., Agbor-Enoh, S Luikart, H., Valantine, H. A., Khush, K. K. and Vlaminck, I. D. (2016) Single-stranded DNA library preparation uncovers the origin and diversity of ultrashort cell-free DNA in plasma. Scientific Reports, 6, 27859.
17. Snyder, M. W., Kircher, M., Hill, A. J., Daza, R. M. and Shendure, J. (2016) Cell-free DNA comprises an in vivo nucleosome footprint that informs its tissues-of-origin. Cell, 164, 57.
18. Ivanov, M., Baranova, A., Butler, T., Spellman, P. and Mileyko, V. (2015) Non-random fragmentation patterns in circulating cell-free DNA reflect epigenetic regulation. BMC Genomics, 16, 51.
19. Garlan, F., Blanchet, B., Kramkimel, N., Puszkiel, A., Golmard, J.-L., Noe, G., Dupin, N., Laurent-Puig, P., Vidal, M., Taly, V., et al. (2017) Circulating Tumor DNA Measurement by Picoliter Droplet-Based Digital PCR and Vemurafenib Plasma Concentrations in Patients with Advanced BRAF-Mutated Melanoma. Targ Oncol, 12, 365-371.
20. Aucamp, J., Bronkhorst, A. J., Badenhorst, C. P. S. and Pretorius, P J. (2016) A historical and evolutionary perspective on the biological significance of circulating DNA and extracellular vesicles. Cell. Mol. Life Sci., 73, 4355-4381.
21. Jahr, S., Hentze, H., Englisch, S., Hardt, D., Fackelmayer, F. O., Hesch, R.-D. and Knippers, R. (2001) DNA Fragments in the Blood Plasma of Cancer Patients: Quantitations and Evidence for Their Origin from Apoptotic and Necrotic Cells. Cancer Res, 61, 1659-1665.
22. Nicolaou, K. C., Li, T., Nakada, M., Hummel, C. W., Hiatt, A. and Wrasidlo, W. (1994) Calicheamicin θ1 I: A rationally designed molecule with extremely potent and selective DNA cleaving properties and apoptosis inducing activity. ANGEW CHEM (INT ED ENGL), 33, 183-186.
23. Négrier, S., Escudier, B., Gomez, F., Douillard, J.-Y., Ravaud, A., Chevreau, C., Buclon, M., Pérol, D. and Lasset, C. (2002) Prognostic factors of survival and rapid progression in 782 patients with metastatic renal carcinomas treated by cytokines: a report from the Groupe Français d'Immunothérapie. Ann Oncol, 13, 1460-1468.
24. Galon, J., Pages, F., Mlecnik, B. and BINDEA, G. (2014) Methods and kits for screening patients with a cancer.
25. Restifo, N. P., Dudley, M. E. and Rosenberg, S. A. (2012) Adoptive immunotherapy for cancer: harnessing the T cell response. Nat Rev Immunol, 12, 269-281.
26. Rosenberg, S. A., Packard, B. S., Aebersold, P. M., Solomon, D., Topalian, S. L., Toy, S. T., Simon, P., Lotze, M. T., Yang, J. C., Seipp, C. A., et al. (1988) Use of Tumor-Infiltrating Lymphocytes and Interleukin-2 in the Immunotherapy of Patients with Metastatic Melanoma. New England Journal of Medicine, 319, 1676-1680.
27. Rosenberg, S. A., Lotze, M. T., Yang, J. C., Aebersold, P. M., Linehan, W. M., Seipp, C. A. and White, D. E. (1989) Experience with the use of high-dose interleukin-2 in the treatment of 652 cancer patients. Ann Surg, 210, 474-485.
28. Meddeb, R., Pisareva, E. and Thierry, A. R. (2019) Guidelines for the Preanalytical Conditions for Analyzing Circulating Cell-Free DNA. Clinical Chemistry, 65, 623-633.
29. Gansauge, M.-T. and Meyer, M. (2013) Single-stranded DNA library preparation for the sequencing of ancient or damaged DNA. Nat. Protocols, 8, 737-748.
30. Martin, M. (2011) Cutadapt removes adapter sequences from high-throughput sequencing reads. EMBnet.journal, 17, 10-12.
31. Li, H. and Durbin, R. (2009) Fast and accurate short read alignment with Burrows-Wheeler transform. Bioinformatics, 25, 1754-1760.
32. Li, H., Handsaker, B., Wysoker, A., Fennell, T., Ruan, J., Homer, N., Marth, G., Abecasis, G., Durbin, R. and 1000 Genome Project Data Processing Subgroup (2009) The Sequence Alignment/Map format and SAMtools. Bioinformatics, 25, 2078-2079.
33. Bustin, S. A., Benes, V., Garson, J. A., Hellemans, J., Huggett, J., Kubista, M., Mueller, R., Nolan, T., Pfaffl, M. W., Shipley, G. L., et al. (2009) The MIQE Guidelines: Minimum Information for Publication of Quantitative Real-Time PCR Experiments. Clinical Chemistry, 55, 611-622.
34. Thierry A R., S. C. (2017) US Patent Application for METHODS FOR SCREENING A SUBJECT FOR A CANCER Patent Application (Application #17306721.6 issued Dec. 7, 2017).
35. Jiang P, Chan C W1, Chan K C, Cheng S H, Wong J, Wong V W, Wong G L, Chan S L, Mok T S, Chan H L, Lai P B, Chiu R W, Lo Y M. Lengthening and shortening of plasma DNA in hepatocellular carcinoma patients. Proc Natl Acad Sci USA. 2015 Mar. 17; 112(11):E1317-25. doi: 10.1073/pnas.1500076112.
36. Cristiano S et al. Genome-wide cell-free DNA fragmentation in patients with cancer. Nature. 2019 June; 570(7761):385-389. doi: 10.1038/s41586-019-1272-6. Epub 2019 May 29.

1. A method for screening a subject for a cancer and treating the subject for the cancer comprising the steps of:
i. extracting cell free nucleic acids (cfDNA) from a sample obtained from the subject;
ii. determining, within the cfDNA, the level of at least one double stranded DNA fragment having a length from 20 to 440 bp;
iii. comparing the level determined at step ii) with a predetermined reference value and;
iv. treating the subject for the cancer when the level determined at step iii) differs from the predetermined reference value.

2. The method according to claim 1, wherein the level of at least one double stranded DNA fragment determined at step ii) has a length from 20 to 400 base pairs.

3. The method according to claim 1 wherein the at least one double stranded DNA fragment has a length of 120+/−5, 130+/−5 or 134+/−5 bp.

4. The method according to claim 1 wherein the level is calculated with a group of double stranded DNA fragments having a length range of 119 to 120.

5. The method according to claim 2 which comprises the steps of:
i. extracting cell free nucleic acids from the sample obtained from the subject;
ii. determining the level of a first single double stranded DNA fragment having a length from 20 to 440 bp or a first group of double stranded DNA fragments each of which has a length from 20 to 440 bp;
determining the level of a second double stranded DNA fragment having a length from 20 to 440 bp or a second group of double stranded DNA fragments each of which has a length from 20 to 440 bp;
iv. calculating a ratio of the level determined at step ii) to the level determined at step iii) or alternatively the difference of the level determined at step iii) from the level determined at step ii);
v. comparing the ratio or the difference determined at step iv) with a predetermined corresponding reference value and;
vi. treating the subject for the cancer when the ratio determined at step iv) differs from the predetermined corresponding reference value.

6. A method for screening a subject for a cancer and treating the subject for the cancer comprising the steps of:
i. extracting cell free nucleic acids (cfDNA) from a sample obtained from the subject;
dividing cfDNA into samples s1 and s2;
determining the level of at least one double stranded DNA fragment having a length from 20 to 440 bp from sample s1;
iv. denaturating the cf DNA of sample s2 to obtain single stranded DNA fragments and determining the level of at least one of the single stranded DNA fragments having a length from 20 to 440 nt;
v. calculating a ratio of: the level determined at step iii) to the level determined at step iv) or the level determined at step iv) to the level determined at step ii);
vi. comparing the ratio determined at step v) with a predetermined corresponding reference value and;
vii. treating the subject for the cancer when the ratio determined at step v) differs from the predetermined corresponding reference value.

7. The method according to claim 6 which comprises the steps of:
i. extracting cell free nucleic acids (cfDNA) from a sample obtained from the subject;
dividing the cfDNA into 2 samples s1 and s2;
determining the level of at least one double stranded DNA fragment having a length from 20 to 400 bp from sample s1;
iv. denaturating the cf DNA of sample s2 to obtain single stranded DNA fragments and determining the level of at least one of the single stranded DNA fragments having a length from 20 to 400 nt;
v. calculating a difference between the level determined at step iii) to the level determined at step iv);
vi. comparing the difference determined at step v) with a predetermined corresponding reference value and;
vii. treating the subject for the cancer when the difference determined at step v) differs from the predetermined corresponding reference value.

8. The method according to claim 7 wherein the difference is calculated for single stranded DNA fragments having a length of from 110 and 80 bp, 100 and 90 bp, 155 and 125 bp, 90 and 155 bp, 130 and 160 bp, 120 and 150 bp or 120 and 150 bp.

9. The method according to claim 7 wherein the level of a group of double stranded DNA fragments or single stranded DNA fragments is from 70 to 150 bp/nt, 70 to 145 bp/nt or 40 to 145 bp/nt.

10. The methods according to claim 1 wherein the at least one double stranded DNA fragment can has a length of 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; 110; 111; 112; 113; 114; 115; 116; 117; 118; 119; 120; 121; 122; 123; 124; 125; 126; 127; 128; 129; 130; 131; 132; 133; 134; 135; 136; 137; 138; 139; 140; 141; 142; 143; 144; 145, 146; 147; 148; 149; 150; 151; 152; 153; 154; 155; 156; 157; 158; 159; 160; 161; 162; 163; 164; 165; 166; 167; 168; 169; 170; 171; 172; 173; 174; 175; 176; 177; 178; 179; 180; 181; 182; 183; 184; 185; 186; 187; 188; 189; 190; 191; 192; 193; 194; 195; 196; 197; 198; 199; 200; 201; 202; 203; 204; 205; 206; 207; 208; 209; 210; 211; 212; 213; 214; 215; 216; 217; 218; 219; 220; 221; 222; 223; 224; 225; 226; 227; 228; 229; 230; 231; 232; 233; 234; 235; 236; 237; 238; 239; 240; 241; 242; 243; 244; 245; 246; 247; 248, 249; 250; 251; 252; 253; 256; 257; 258; 259; 260; 261; 262; 263; 264; 265; 266; 267; 268; 269; 270; 271; 272; 273; 274; 275; 276; 277; 278; 279; 280; 281; 282; 283; 284; 285; 286; 287; 288; 289; 290; 291; 292; 293; 294; 295; 296; 297; 298; 299; 300; 301; 302; 303; 304; 305; 306; 307; 308; 309; 310; 311; 312; 313; 314; 315; 316; 317; 318; 319; 320; 321; 322; 323; 324; 325; 326; 327; 328; 329; 330; 331; 332; 333; 334; 335; 336; 337; 338; 339; 340; 341; 342; 343; 344; 345; 346; 347; 348; 349; 350; 351; 352; 353; 354; 355; 356; 357; 358; 359; 360; 361; 362; 363; 364; 365; 366; 367; 368; 369; 370; 371; 372; 373; 374; 375; 376; 377; 378; 379; 380; 381; 382; 383; 384; 385; 386; 387; 388; 389; 390; 391; 392; 393; 394; 395; 396; 397; 398; 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439 or 440 bp.

11. The methods according to claim 6, wherein the at least one single stranded DNA fragment has a length of 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; 110; 111; 112; 113; 114; 115; 116; 117; 118; 119; 120; 121; 122; 123; 124; 125; 126; 127; 128; 129; 130; 131; 132; 133; 134; 135; 136; 137; 138; 139; 140; 141; 142; 143; 144; 145, 146; 147; 148; 149; 150; 151; 152; 153; 154; 155; 156; 157; 158; 159; 160; 161; 162; 163; 164; 165; 166; 167; 168; 169; 170; 171; 172; 173; 174; 175; 176; 177; 178; 179; 180; 181; 182; 183; 184; 185; 186; 187; 188; 189; 190; 191; 192; 193; 194; 195; 196; 197; 198; 199; 200; 201; 202; 203; 204; 205; 206; 207; 208; 209; 210; 211; 212; 213; 214; 215; 216; 217; 218; 219; 220; 221; 222; 223; 224; 225; 226; 227; 228; 229; 230; 231; 232; 233; 234; 235; 236; 237; 238; 239; 240; 241; 242; 243; 244; 245; 246; 247; 248, 249; 250; 251; 252; 253; 256; 257; 258; 259; 260; 261; 262; 263; 264; 265; 266; 267; 268; 269; 270; 271; 272; 273; 274; 275; 276; 277; 278; 279; 280; 281; 282; 283; 284; 285; 286; 287; 288; 289; 290; 291; 292; 293; 294; 295; 296; 297; 298; 299; 300; 301; 302; 303; 304; 305; 306; 307; 308; 309; 310; 311; 312; 313; 314; 315; 316; 317; 318; 319; 320; 321; 322; 323; 324; 325; 326; 327; 328; 329; 330; 331; 332; 333; 334; 335; 336; 337; 338; 339; 340; 341; 342; 343; 344; 345; 346; 347; 348; 349; 350; 351; 352; 353; 354; 355; 356; 357; 358; 359; 360; 361; 362; 363; 364; 365; 366; 367; 368; 369; 370; 371; 372; 373; 374; 375; 376; 377; 378; 379; 380; 381; 382; 383; 384; 385; 386; 387; 388; 389; 390; 391; 392; 393; 394; 395; 396; 397; 398; 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439 or 440 nt.

12. (canceled)

13. A method for screening a subject for a cancer and treating the subject for the cancer comprising the steps of:
i. extracting the cell free nucleic acids (cfDNA) from a sample obtained from the subject;
ii. dividing the cfDNA into 2 samples s1 and s2;
iii. determining a level of double stranded DNA fragments having a length from 20 to 400 bp from sample s1;
iv. denaturating the cf DNA of sample s2 to obtain single stranded DNA fragments and determining a level of single stranded DNA fragments having a length from 20 to 400 nt;
v. plotting a cumulative size frequency obtained from sample s1 and sample s2 from cumulative size frequency reference values;
vi. plotting a difference of ΔS values from sample s1 and sample s2 obtained in step v) and determining a gain and a loss of fragments;
vii. comparing the gain and the loss determined at step vi) with a predetermined corresponding reference value and;
viii. treating the subject for the cancer when the gain or the loss determined at step vi) differs from the predetermined corresponding reference value.

14. A method for screening a subject for a cancer and treating the subject for the cancer comprising the steps of:
i. extracting cell free nucleic acids (cfDNA) from a sample obtained from the subject;
ii. dividing the cfDNA into 2 samples s1 and s2;
iii. determining a level of double stranded DNA fragments having a length from 20 to 440 bp from sample s1;
iv. denaturating the cf DNA of sample s2 to obtain single stranded DNA fragments and determining a level of single stranded DNA fragments having a length from 20 to 440 nt;
v. plotting a cumulative size frequency obtained from sample s1 and sample s2 from cumulative size frequency reference values;
vi. plotting a difference of the ΔS values from sample s1 and sample s2 obtained in step v) and determining a gain and a loss of fragments;
vii. comparing the gain and the loss determined at step vi) with a predetermined corresponding reference value and;
viii. treating the subject for the cancer when the gain or the loss determined at step vi) differs from the predetermined corresponding reference value.

15. (canceled)

16. (canceled)
